(19) United States
(12) Patent Application Publication
(10) Pub. No.: US 2025/0120959 A1
(43) Pub. Date: Apr. 17, 2025

(54) NEW FORMULATIONS OF PITOLISANT AND METHODS OF USE

(71) Applicant: Bioprojet Pharma, Paris (FR)

(72) Inventors: Marc CAPET, Melesse (FR); Philippe ROBERT, Grenoble (FR); Olivier FINANCE, Rennes (FR); Jeanne-Marie LECOMTE, Paris (FR); Jean-Charles SCHWARTZ, Paris (FR); Elsie MELSOPP, Wilmington, NC (US); Kumaraswamy BUDUR, Chicago, IL (US)

(21) Appl. No.: 18/917,144

(22) Filed: Oct. 16, 2024

Related U.S. Application Data
(60) Provisional application No. 63/590,678, filed on Oct. 16, 2023.
(60) Provisional application No. 63/616,039, filed on Dec. 29, 2023.
(60) Provisional application No. 63/679,270, filed on Aug. 5, 2024.

(30) Foreign Application Priority Data
Jul. 18, 2024 (EP) 24306216.3

(51) Int. Cl. A61K 31/4453; A61K 9/20; A61K 9/28

(52) U.S. Cl. CPC A61K 31/4453; A61K 9/2009; A61K 9/2013; A61K 9/2018; A61K 9/2054; A61K 9/2095; A61K 9/2846; A61K 9/2853; A61K 9/2866; A61K 9/2893

(57) ABSTRACT

The present disclosure relates generally to dosage forms (e.g., oral dosage forms) and pharmaceutical compositions comprising a pharmaceutically active agent comprising pitolisant or a pharmaceutically acceptable salt, solvate, or hydrate thereof, an alkaline agent, a delayed-release coating, and optionally one or more pharmaceutically acceptable excipients; as well as methods of using the dosage forms and pharmaceutical compositions, and methods of making the same.

### RELATED APPLICATIONS

This application claims the benefit of U.S. Provisional Application No. 63/590,678, filed on Oct. 16, 2023, U.S. Provisional Application No. 63/616,039, filed on Dec. 29, 2023, U.S. Provisional Application No. 63/679,270, filed on Aug. 5, 2024, and European Patent Application No. 24306216.3, filed on Jul. 18, 2024, the entire contents of which are each incorporated herein by reference.

### BACKGROUND

Pitolisant formulations are useful to treat many diseases and disorders, particularly sleep disorders such as excessive daytime sleepiness (EDS) and cataplexy. For example, WAKIX® (pitolisant monohydrochloride; Harmony Biosciences, LLC of Plymouth Meeting, PA) is a prescription medicine approved for the treatment of EDS or cataplexy in adults with narcolepsy, and for the treatment of EDS in pediatric patients 6 years of age and older with narcolepsy.

Existing pitolisant formulations are usually administered with a titrated dose in the first two weeks, beginning with around 8.9 mg/day that is doubled after one week, and then doubled again after a second week to reach a dose of 35.6 mg/day, which may be lowered depending on the patient's tolerance.

### SUMMARY

The present disclosure relates generally to dosage forms (e.g., oral dosage forms) and pharmaceutical compositions comprising a pharmaceutically active agent comprising pitolisant, which is represented by the structure of Formula (I):

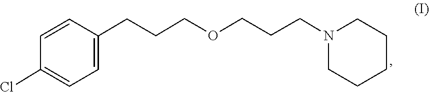 [SMILES: Clc1ccc(CCCOCCCN2CCCCC2)cc1]

or a pharmaceutically acceptable salt, solvate, or hydrate thereof, an alkaline agent, a delayed release (e.g., enteric) coating, and optionally one or more pharmaceutically acceptable excipients. Also disclosed are methods of using the dosage forms and pharmaceutical compositions (e.g., for treating a disease or disorder), as well as methods of making the same. The dosage form may further comprise an anti-moisture barrier.

An advantage of the dosage forms and pharmaceutical compositions of the present disclosure is that they can have a faster Tₘₐₓ and greater area under the curve (AUC) compared to an equivalent pharmaceutical composition that lacks a base (e.g., an alkaline agent) and does not have an enteric coating, such as WAKIX®.

An advantage of the dosage forms and pharmaceutical compositions of the present disclosure is that they can have reduced or no gastric side-effects, such as nausea, emesis, and upset stomach, when administered (e.g., orally). For example, a subject that receives by oral administration a dosage form or pharmaceutical composition disclosed herein may experience minimal or no side-effects such as nausea, upset stomach, or emesis, relative to when the subject is orally administered an equivalent non-enterically coated dosage form or pharmaceutical composition, such as WAKIX®.

Another advantage of the dosage forms and pharmaceutical compositions of the present disclosure is they can result in improved patient compliance in the subjects receiving them, relative to patient compliance typically seen in subjects receiving a non-enterically coated dosage forms or pharmaceutical compositions, such as WAKIX®. This is due in part to the reduced side-effects experienced by subjects receiving the dosage forms or pharmaceutical compositions of the present disclosure, e.g., following oral administration. As the side-effects experienced by subjects receiving the dosage forms or pharmaceutical compositions disclosed herein may be minimal or non-existent, subjects are more likely to comply with the titration period and/or prescribed dosage regimen, and will be more likely to continue at the maximum recommended dosage, therefore receiving full clinical benefit with minimal or no side-effects.

Additionally, and without wishing to be bound by theory, it is believed that certain dosage forms and pharmaceutical compositions disclosed herein can deliver an optimized pharmacokinetic profile, compared to when using a conventional dose form comprising pitolisant (e.g., WAKIX®). Indeed, data provided herein demonstrates that dose forms and pharmaceutical compositions of the present disclosure significantly increased relative bioavailability, and decreased variability, compared to WAKIX®. Further, certain dosage forms and pharmaceutical compositions can safely deliver a higher dose of pitolisant (e.g., up to about 180 mg per day) compared to WAKIX®, which is approved for doses up to 35.6 mg per day.

It is further believed that dose forms and pharmaceutical compositions of the present disclosure will not only have improved pharmacokinetic properties, but the presence of an enteric coating may also contribute to better tolerability, and will allow subjects to begin treatment without titrating the dose. This is notable, as it is believed as many as 90% of subjects with narcolepsy experience gastrointestinal symptoms such as nausea, dyspepsia, and abdominal discomfort, which may be due to the orexin deficiency that is present in subjects with narcolepsy, because orexin has effects on the vagus nerve in the brain, which is the central controller of gut motility. Further, not only are subjects with narcolepsy predisposed to gastrointestinal issues, but gastrointestinal side effects may also be caused by common narcolepsy treatments. Thus, the dose forms and pharmaceutical compositions disclosed herein can help to address the predisposition to gastrointestinal issues in patients with narcolepsy, as well as offer an alternative to subjects that have experienced gastrointestinal issues linked with other narcolepsy treatments.

Also, without wishing to be bound by theory, it is believed that certain other dosage forms and pharmaceutical compositions disclosed herein are bioequivalent to counterpart dosage forms comprising the same pharmaceutically active agent, without an enteric coating, such as WAKIX®.

Accordingly, it is believed that the dose forms and pharmaceutical compositions of the present disclosure, with optimized pharmacokinetic profiles and enteric coatings, may address significant unmet need in subjects with disorders, such as fatigue, excessive daytime sleepiness (EDS), cataplexy, narcolepsy, sleep apnea (e.g., obstructive sleep apnea), sleep induced apnea, or diurnal somnolence. It is also believed that the dose forms and pharmaceutical compositions of the present disclosure may also be used to safely provide higher doses of pitolisant relative to currently available compositions of pitolisant (e.g., WAKIX®).

In some aspects the present disclosure relates to an oral dosage form comprising (e.g., consisting essentially of, e.g., consisting of): a core; and a delayed-release (e.g., enteric) coating that surrounds the core, wherein the core comprises pitolisant or a pharmaceutically acceptable salt, solvate, or hydrate thereof, and an alkaline agent; and optionally one or more pharmaceutically acceptable excipients. The core may comprise pitolisant monohydrochloride (e.g., crystalline pitolisant monohydrochloride). The alkaline agent may comprise magnesium carbonate, potassium carbonate, or sodium carbonate. An alkaline agent may sometimes be referred to herein as a base.

The delayed-release (e.g., enteric) coating can comprise a polymer. The polymer may comprise an ionizable functional group, such as a carboxylic acid group. The delayed-release (e.g., enteric) coating can comprise a polymer such as a cellulosic material (e.g., an alkylcellulose), an acrylic polymer, an acrylate polymer, a methacrylic polymer, a methacrylate polymer, or a methacrylate copolymer (e.g., an anionic methacrylate copolymer). For example, the delayed-release coating may comprise ACRYL-EZE® aqueous acrylic enteric system from Colorcon, Inc. (275 Ruth Road, Harleysville PA 19438).

The delayed-release coating may further comprise a plasticizer, such as polyethylene glycol (PEG), e.g., PEG8000.

The dosage forms of the present disclosure may comprise an anti-moisture barrier. The anti-moisture barrier can be positioned between the core and the enteric coating. For example, the dosage form may comprise a core tablet that is coated with the anti-moisture barrier (to give an anti-moisture barrier coated tablet), which itself is further coated with an enteric coating (to give an enteric-coated tablet). The anti-moisture barrier may comprise an OPADRY® polymer, such as OPADRY® amb II polyvinyl alcohol-based coating from Colorcon, Inc.

The dosage form can comprise between about 1 mg and about 400 mg pitolisant or a pharmaceutically acceptable salt, solvate, or hydrate thereof (e.g., between about 1 mg and about 250 mg, or between about 20 mg and about 200 mg, e.g., about 10 mg, about 20 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, or about 300 mg). The dosage form can comprise between about 1 mg and about 80 mg pitolisant or a pharmaceutically acceptable salt, solvate, or hydrate thereof (e.g., between about 1 mg and about 70 mg, between about 1 mg and about 60 mg, between about 1 mg and about 50 mg, between about 1 mg and about 40 mg, between about 20 mg and about 40 mg, between about 40 mg and about 60 mg, or between about 50 mg and about 70 mg). For example, the dosage form can comprise about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, or about 75 mg of pitolisant or a pharmaceutically acceptable salt, solvate, or hydrate thereof. The pitolisant or pharmaceutically acceptable salt, solvate, or hydrate thereof, may be contained within the core of the dose form, and may not be present in the delayed release coating or in the anti-moisture barrier. For example, a dosage form of the present disclosure may comprise about 40 mg of pitolisant or a pharmaceutically acceptable salt, solvate, or hydrate thereof (e.g., 40 mg of pitolisant monohydrochloride).

The dosage form can comprise between about 1 mg and about 40 mg of an alkaline agent (e.g., magnesium carbonate, potassium bicarbonate, or sodium carbonate). For example, the dosage form can comprise about 1 mg, about 2 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg of a base. For example, the dosage form can comprise about 1 mg, about 2 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg of magnesium carbonate. For example, the dosage form can comprise about 1 mg, about 2 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg of potassium bicarbonate. For example, the dosage form can comprise about 1 mg, about 2 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg of sodium carbonate. The alkaline agent may be contained within the core of the dose form, and may not be present in the delayed release coating or in the anti-moisture barrier.

The dosage form can comprise a pharmaceutically acceptable excipient. For example, the pharmaceutically acceptable excipient may be microcrystalline cellulose, lactose (e.g., anhydrous lactose), croscarmellose sodium, magnesium stearate, or colloidal silica (e.g., anhydrous colloidal silica). For example, a dosage form of the present disclosure can comprise a core, wherein the core comprises one or more of microcrystalline cellulose, lactose (e.g., anhydrous lactose), croscarmellose sodium, magnesium stearate, or colloidal silica (e.g., anhydrous colloidal silica).

The dosage form of the present disclosure can comprise crystalline pitolisant monohydrochloride. The dosage form may further comprise a pharmaceutically acceptable excipient. In some embodiments, the dosage form comprises a core, wherein the core comprises (e.g., consists essentially of, e.g., consists of) pitolisant monohydrochloride, an alkaline agent (e.g., magnesium carbonate, potassium bicarbonate, or sodium carbonate), microcrystalline cellulose, lactose (e.g., anhydrous lactose), croscarmellose sodium, magnesium stearate, and colloidal silica (e.g., anhydrous colloidal silica).

The core of the dosage form disclosed herein can be surrounded by an anti-moisture barrier that comprises (e.g., consists essentially of, e.g., consists of) a PVA-based polymer, e.g., OPADRY® amb II. The core surrounded by the anti-moisture barrier may further be surrounded by an enteric coating that comprises (e.g., consists essentially of, e.g., consists of) a copolymer of methacrylic acid and ethyl acrylate, e.g., ACRYL-EZE®.

The pitolisant or pharmaceutically acceptable salt, solvate, or hydrate thereof included in a dosage form of the present disclosure can have an X-ray diffractogram that comprises characteristic peaks (2θ) at 11.2°, 19.9°, 20.7°, and 34.1° (0.2°). For example, the pitolisant or pharmaceutically acceptable salt, solvate, or hydrate thereof can have an X-ray diffractogram that comprises characteristic peaks (2θ) at 11.2°, 15.4°, 16.3°, 16.9°, 17.8°, 19.9°, 20.7°, 21.0°, 21.8°, 22.6°, 24.5°, 24.6°, 25.0°, 25.5°, 26.3°, 28.3°, 30.3°, 34.1°, 35.8°, 40.0°, and 46.0° (±0.2°). An exemplary X-ray diffractogram is provided in FIG. 1. In some embodiments, the pitolisant or pharmaceutically acceptable salt, solvate, or hydrate thereof present in a dosage form or pharmaceutical composition of the present disclosure has an X-ray diffractogram substantially the same as that provided in FIG. 1.

An oral dosage form of the present disclosure may be a tablet.

A dosage form of the present disclosure may be bioequivalent to a dosage form comprising pitolisant or a pharmaceutically acceptable salt, solvate, or hydrate thereof, in about the same amount, that does not comprise an enteric coating (e.g., WAKIX®).

A dosage form of the present disclosure may have superior bioavailability of pitolisant as compared to a dosage form comprising pitolisant or a pharmaceutically acceptable salt, solvate, or hydrate thereof, in about the same amount, that does not comprise an enteric coating and/or an alkaline agent in the core (e.g., WAKIX®). For example, a dosage form of the present disclosure may have a bioavailability of pitolisant that is about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 50%, about 60%, about 70%, about 80%, about 90%, or about 100%, or greater, relative to the bioavailability provided by WAKIX®.

The present disclosure also relates to methods of treating a disease or disorder, comprising orally administering to a subject in need thereof an oral dosage form of the present disclosure. The disease or disorder can be fatigue. The disease or disorder can be a sleep disorder. For example, the disease or disorder can be fatigue, excessive daytime sleepiness (EDS), cataplexy, narcolepsy, sleep apnea (e.g., obstructive sleep apnea), sleep induced apnea, diurnal somnolence, idiopathic hypersomnia (IH), or sleep inertia (e.g., sleep inertia in IH). For example, the disease or disorder can be excessive daytime sleepiness (EDS), cataplexy, narcolepsy, sleep apnea (e.g., obstructive sleep apnea), sleep induced apnea, or diurnal somnolence. In some embodiments, the disease or disorder is excessive daytime sleepiness (EDS). In some embodiments, the disease or disorder is cataplexy. In some embodiments, the method involves treating a disease or disorder in a subject that has narcolepsy (e.g., an adult with narcolepsy). In some embodiments, the disease or disorder is idiopathic hypersomnia (IH). In some embodiments, the disease or disorder is sleep inertia (e.g., sleep inertia in IH).

The present disclosure further relates to a dosage form (e.g., oral dosage form) described herein for use in the treatment of a disease or disorder, optionally, wherein the disease or disorder is fatigue, excessive daytime sleepiness (EDS), cataplexy, narcolepsy, sleep apnea (e.g., obstructive sleep apnea), sleep induced apnea, diurnal somnolence, idiopathic hypersomnia (IH), or sleep inertia (e.g., sleep inertia in IH). The disease or disorder may be in a subject with narcolepsy (e.g., an adult subject with narcolepsy).

The present disclosure also relates to use of an oral dosage form described herein for the manufacture of a medicament for the treatment of a disease or disorder, optionally, wherein the disease or disorder is fatigue, excessive daytime sleepiness (EDS), cataplexy, narcolepsy, sleep apnea (e.g., obstructive sleep apnea), sleep induced apnea, diurnal somnolence, idiopathic hypersomnia (IH), or sleep inertia (e.g., sleep inertia in IH). The disease or disorder may be in a subject with narcolepsy (e.g., an adult subject with narcolepsy).

The present disclosure also relates to a method of making an oral dosage form described herein. The method can comprise the steps of: (a) blending pitolisant or a pharmaceutically acceptable salt, solvate, or hydrate thereof, and an alkaline agent (e.g., magnesium carbonate, potassium bicarbonate, or sodium carbonate), with one or more pharmaceutically acceptable excipients (e.g., microcrystalline cellulose, lactose (e.g., anhydrous lactose), croscarmellose sodium, magnesium stearate, colloidal silica (e.g., anhydrous colloidal silica) or a combination thereof), to provide a blend; (b) tableting the blend (e.g., using a tableting press) to provide a core tablet; (c) optionally, coating the core tablet with an anti-moisture barrier, to provide an anti-moisture coated tablet; and (d) coating the core tablet of step (b) or the anti-moisture barrier coated tablet of step (c) with a delayed-release (e.g., enteric) coating, to provide a delayed release coated tablet.

The anti-moisture barrier optionally applied in step I may comprise a polymer, such as an OPADRY® polymer, e.g., OPADRY® amb II.

The enteric coating applied in step (d) may also comprise a polymer, such as a copolymer of methacrylic acid and ethyl acrylate, e.g., ACRYL-EZE®. The enteric coating applied in step (d) may also comprise a plasticizer, such as triethyl citrate or PEG (e.g., PEG8000).

### BRIEF DESCRIPTION OF DRAWINGS

FIG. 1 shows an X-ray diffraction pattern of a polymorph of crystalline pitolisant monohydrochloride suitable for use in a dosage form of the present disclosure.

FIG. 2 shows a graph illustrating the permeability coefficient (Papp) of five different pitolisant hydrochloride formulations containing an alkaline agent.

FIG. 3 is a graph illustrating the cumulative Papp (AUC 0-120 min) of five different pitolisant hydrochloride formulations containing an alkaline agent.

FIG. 4 is a graph illustrating the effect of pH on the permeability coefficient (Papp) of pitolisant hydrochloride formulations.

FIG. 5 is a graph illustrating the cumulative Papp (AUC 0-120 min) of fifteen different pitolisant hydrochloride formulations (40 mg) containing an alkaline agent.

FIG. 6 is a graph illustrating the apparent permeability of five different pitolisant hydrochloride (40 mg) formulations containing an alkaline agent (30 mg).

FIGS. 7A and 7B are graphs illustrating the arithmetic mean pitolisant serum concentrations vs. theoretical time profiles by treatment (FIG. 7A is linear scale, FIG. 7B is log-linear scale).

FIGS. 8A and 8B are graphs (Spaghettis plot) illustrating the individual pitolisant serum concentrations vs. actual time profiles with Treatment A (FIG. 8A is linear scale; FIG. 8B is log-linear scale).

FIGS. 9A and 9B are graphs (Spaghettis plot) illustrating the individual pitolisant serum concentrations vs. actual time profiles with Treatment B (FIG. 9A is linear scale; FIG. 9B is log-linear scale).

FIGS. 10A and 10B are graphs (Spaghettis plot) illustrating the individual pitolisant serum concentrations vs. actual time profiles with Treatment C (FIG. 10A is linear scale; FIG. 10B is log-linear scale).

FIGS. 11A and 11B are graphs (Spaghettis plot) illustrating the individual pitolisant serum concentrations vs. actual time profiles with Treatment D (FIG. 11A is linear scale; FIG. 11B is log-linear scale).

FIGS. 12A and 12B are graphs (Spaghettis plot) illustrating the individual pitolisant serum concentrations vs. actual time profiles with Treatment E (FIG. 12A is linear scale; FIG. 12B is log-linear scale).

### DETAILED DESCRIPTION

The present disclosure relates generally to dosage forms (e.g., oral dosage forms) and pharmaceutical compositions comprising a pharmaceutically active agent comprising pitolisant, which is represented by Formula (I):

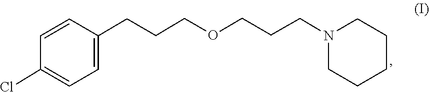 [SMILES: Clc1ccc(CCCOCCCN2CCCCC2)cc1]

or a pharmaceutically acceptable salt, solvate, or hydrate thereof, an enteric coating, an alkaline agent, and optionally one or more pharmaceutically acceptable excipients, and optionally an anti-moisture barrier coating.

The dosage forms and pharmaceutical compositions of the present disclosure, which comprise a core of pitolisant and an alkaline agent (e.g., magnesium carbonate, potassium bicarbonate, or sodium carbonate), covered by a delayed release (e.g., enteric) coating, have many advantages over existing pitolisant formulations such as WAKIX®. This combination results in better pharmacokinetics and pharmacodynamics (e.g., faster Tₘₐₓ, or greater AUC). In particular, they can be better tolerated by patients, owing to less severe or no side-effects (e.g., gastric side effects, such as stomach upset, nausea, and emesis), relative to non-enterically coated pitolisant dosage forms or pharmaceutical compositions, such as WAKIX®. Consequently, subjects receiving the dosage forms and pharmaceutical compositions of the present disclosure demonstrate better patient compliance relative to subjects receiving pitolisant formulations that do not comprise an enteric coating (e.g., WAKIX®). Another advantage of the dosage forms and pharmaceutical compositions of the present disclosure is that they have superior bioavailability of pitolisant, as compared to WAKIX®, a pitolisant-containing dosage form approved for the treatment of EDS and cataplexy in adult subjects with narcolepsy; or in some cases, certain dosage forms and pharmaceutical compositions of the present disclosure are bioequivalent to WAKIX®, a pitolisant-containing dosage form approved for the treatment of EDS and cataplexy in adult subjects with narcolepsy, which is unexpected.

### Definitions

The articles “a” and “an” are used herein to refer to one or more than one (i.e., to at least one) of the grammatical object of the article. By way of example, “an element” means one element or more than one element.

The term “about” when referring to a measurable value such as an amount, a temporal duration, and the like, is meant to encompass variations of ±20% or less, or in some instances ±15% or less, or in some instances ±10% or less, or in some instances ±5% or less, or in some instances ±1% or less, or in some instances ±0.1% or less, from the specified value, as such variations are appropriate.

The phrase “and/or” as used herein should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “administer,” “administering,” or “administration,” as used herein refer to implanting, absorbing, ingesting, injecting, inhaling, or otherwise introducing a compound, dosage form, or pharmaceutical composition.

The terms “comprise,” “comprises,” and “comprising” are used herein in a non-exclusive sense, except where the context requires otherwise. Likewise, the term “include” and its grammatical variants are intended to be non-limiting, such that recitation of items in a list is not to the exclusion of other like items that can be substituted or added to the listed items.

The term “effective amount” or a “therapeutically effective amount” as used herein refers to an amount of a compound, a dosage form, or a pharmaceutical composition, described herein which is sufficient to achieve a desired result under the conditions of administration. For example, an effective amount of a compound, dosage form, or pharmaceutical composition disclosed herein for treating excessive sleep disorder (EDS), e.g., in a subject with narcolepsy, is an amount that can reduce the effects of the EDS, and/or reduce or eliminate the severity of a symptom associated with the EDS. A skilled clinician can determine appropriate dosing based on a variety of considerations including the severity of the disease, the subject's age, weight, general health and other considerations. A dosage form or pharmaceutical composition disclosed herein may be administered to provide an amount of about 0.01 mg to about 400 mg (e.g., about 0.1 mg to about 200 mg) of a pharmaceutically active agent (e.g., pitolisant or a pharmaceutically acceptable salt, solvate, or hydrate thereof), e.g., about 1 mg, about 2 mg, about 3 mg, about 4 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 120 mg, about 140 mg, about 160 mg, about 180 mg, about 200 mg, about 250 mg, or greater. In some aspects, a dosage form or pharmaceutical composition of the present disclosure is administered to provide about 5 mg pitolisant hydrochloride. In some aspects, a dosage form or pharmaceutical composition of the present disclosure is administered to provide about 20 mg pitolisant hydrochloride. In some aspects, a dosage form or pharmaceutical composition of the present disclosure is administered to provide about 40 mg pitolisant hydrochloride. In some aspects, a dosage form or pharmaceutical composition of the present disclosure is administered to provide about 60 mg pitolisant hydrochloride. In some aspects, a dosage form or pharmaceutical composition of the present disclosure is administered to provide about 70 mg pitolisant hydrochloride. In some aspects, a dosage form or pharmaceutical composition of the present disclosure is administered to provide about 67 mg pitolisant hydrochloride.

The term “pharmaceutically acceptable excipient” as used herein refers to a non-toxic material that may be formulated with a compound disclosed herein to provide a pharmaceutical composition. Preferably, the pharmaceutically acceptable excipient is inert and does not interfere with the pharmacological activity of a compound which it is formulated with. Pharmaceutically acceptable excipients useful in the manufacture of the pharmaceutical compositions disclosed herein are any of those well known in the art, and include without limitation, diluents, dispersing agents, granulating agents, surface active agents, emulsifiers, disintegrating agents (sometimes referred to herein as disintegrants), binding agents (sometimes referred to herein as binders), flowing agents, preservatives, buffering agents (sometimes referred to herein as buffers), lubricating agents (sometimes referred to herein as lubricants), glidants, adjuvants, fillers, wetting agents, suspending agents, solvents, dispersion media, ion exchangers, salts, electrolytes, waxes, coloring agents, and/or oils, and the like.

For example, a pharmaceutically acceptable excipient may be alumina, a phosphate (e.g., calcium phosphate, dicalcium phosphate, tricalcium phosphate, disodium hydrogen phosphate, potassium hydrogen phosphate), a sulfate (e.g., calcium sulfate), a cellulose (including, e.g., cellulose derivatives, microcrystalline cellulose (including spray-dried microcrystalline cellulose), silicified microcrystalline cellulose, hydroxypropylmethylcellulose, methylcellulose, carboxymethylcellulose or salts thereof (e.g., sodium carboxymethylcellulose, or calcium carboxymethylcellulose)), kaolin, bentonite, VEEGUM®, lactose (e.g., anhydrous lactose, spray dried lactose, hydrated lactose), mannitol, sorbitol, sucrose, inositol, compressible sugar, trehalose, xylitol, acacia, gelatin, glucose, maltodextrin, starch (e.g., corn starch, potato starch), sodium starch glycolate, starch derivatives, an amino acid (e.g., glycine or leucine), magnesium carbonate, polyvinylpyrrolidone (PVP, povidone) (e.g., crosslinked PVP, crospovidone), polyvinyl alcohol, tragacanth, polyethylene glycol, polymethacrylates, mineral clay powders, croscarmellose, poloxamer, fatty acids or salts thereof (e.g., lauric acid, sodium lauryl sulfate, stearic acid, calcium stearate, magnesium stearate, aluminum stearate, oleic acid), hydrogenated vegetable oils, talc, titanium dioxide, glyceryl behenate, silicon dioxide (e.g., colloidal silicon dioxide), a silicate salt (e.g., magnesium trisilicate), lecithin, serum protein (e.g., human serum albumin), sorbic acid, potassium sorbate, a metal cation salt (e.g., a sodium salt, such as sodium chloride, a potassium salt, such as potassium chloride, a magnesium salt, such as or magnesium chloride, a zinc salt, such as zinc chloride), water, dimethylacetamide, protamine sulfate, a polyacrylate, wool fat, ethylenediaminetetraacetic acid (EDTA), a cyclodextrin (e.g., CAPTISOL©), KOLLIDON® CL, CELLACTOSE®, LUDIPRESS®, polysorbates (e.g., TWEEN®, e.g., TWEEN® 20 or TWEEN® 80), and combinations thereof.

The term “pharmaceutically acceptable salt” as used herein refers to a salts of a compound prepared with relatively nontoxic acids or bases, depending on the particular substituents found on the respective compound. When compounds relating to the present disclosure contain relatively basic functionalities (e.g., as in Formula (I)), acid addition salts can be obtained by contacting the neutral form of such compounds with a sufficient amount of the desired acid, either neat or in a suitable solvent (e.g., an inert solvent). For example, the neutral form of pitolisant may be contacted with gaseous hydrochloric acid to provide pitolisant monohydrochloride, which can be present in a dosage form disclosed herein. Examples of pharmaceutically acceptable acid addition salts include those derived from inorganic acids like hydrochloric acid, hydrobromic acid, nitric acid, carbonic acid, monohydrogencarbonic acid, phosphoric acid, monohydrogenphosphoric acid, dihydrogenphosphoric acid, sulfuric acid, monohydrogensulfuric acid, hydriodic acid, or phosphorous acids and the like, as well as the salts derived from organic acids like acetic acid, propionic acid, isobutyric acid, maleic acid, malonic acid, benzoic acid, succinic acid, suberic acid, fumaric acid, lactic acid, mandelic acid, pamoic acid, phthalic acid, benzenesulfonic acid, p-toluenesulfonic acid, citric acid, tartaric acid, methanesulfonic acid, oxalic acid, and the like. Also included are salts of amino acids such as arginate and the like, and salts of organic acids like glucuronic or galacturonic acids and the like. Other pharmaceutically acceptable salts known to those of skill in the art are suitable for pharmaceutical compositions relating to the present disclosure.

The term “solvate” as used herein refers to forms of a compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. Compounds of the present disclosure may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” as used herein refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound and wherein x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “subject” as used herein refers to any animal, such as any mammal, including but not limited to, humans, non-human primates, rodents, dogs, and the like. Non-human primates include chimpanzees, cynomolgus monkeys, spider monkeys, and macaques (e.g., Rhesus). Rodents include mice, rats, woodchucks, ferrets, rabbits, and hamsters. Domestic and game animals include cows, horses, pigs, deer, bison, buffalo, feline species (e.g., domestic cat), canine species (e.g., dog, fox, wolf), avian species, and fish. In some embodiments, the subject is a mammal (e.g., a human, a rat, or a mouse). The subject can be male or female. The subject may be of any age, including an elderly human subject (e.g., 65 years or older), a human subject that is not elderly (e.g., less than 65 years old), or a human pediatric subject (e.g., less than 18 years old). In preferred aspects, the subject is a human.

As used herein, the terms “treat,” “treatment,” “treating,” or grammatically related terms, refer to a method of reducing the effects of a disease or disorder. As is readily appreciated in the art, full eradication of the disease, disorder, or symptoms thereof is preferred but not a requirement for treatment. Desirable effects of treatment include, but are not limited to, preventing occurrence or recurrence of the disease or disorder, alleviation of symptoms, diminishment of any direct or indirect pathological consequences of the disease or disorder, or other improvement of any sign, symptom, or consequence of the disease or disorder, such as prolonged survival, less morbidity, and/or a lessening of side effects.

Throughout this disclosure, various embodiments can be presented in a range format (e.g., from X-Y). It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 5, from 1 to 4, from 1 to 3, from 2 to 6, from 2 to 4, from 3 to 6, etc., as well as individual numbers within that range, e.g., 1, 2, 2.8, 3, 3.6, 4, 5, 5.4, and 6. As another example, a range such as 95-99% includes 95%, 96%, 97%, 98%, or 99% and all subranges such as 96-99%, 96-98%, 96-97%, 97-99%, 97-98%, etc. This applies regardless of the breadth of the range.

All publications (e.g., scientific journal articles, patent publications, and the like) cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

Compounds (e.g., pharmaceutically active agents) disclosed herein may also comprise one or more isotopic substitutions. For example, hydrogen (H) may be in any isotopic form, including ¹H, ²H (D or deuterium), ³H (T or tritium); carbon (C) may be in any isotopic form, including ¹²C, ¹³C, and ¹⁴C; oxygen (O) may be in any isotopic form, including ¹⁶O and ¹⁸O; nitrogen (N) may be in any isotopic form, including ¹⁴N and ¹⁵N; and chlorine (Cl) may be in any isotopic form, including ³⁵Cl and ³⁷Cl.

Various embodiments of the compounds, dosage forms, pharmaceutical compositions, and methods herein are described in further detail below, and additional definitions may be provided throughout the specification.

### Dosage Forms and Pharmaceutical Compositions

Disclosed herein are dosage forms (e.g., oral dosage forms) and pharmaceutical compositions comprising a pharmaceutically active agent, an alkaline agent, and a delayed-release (enteric) coating, and optionally one or more pharmaceutically acceptable excipients. The pharmaceutically active agent is pitolisant, represented by Formula (I):

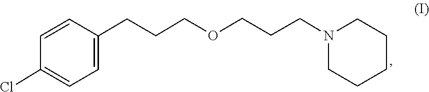 [SMILES: Clc1ccc(CCCOCCCN2CCCCC2)cc1]

or a pharmaceutically acceptable salt, solvate, or hydrate thereof.

The dosage form or pharmaceutical composition can comprise a therapeutically effective amount of the pharmaceutically active agent. For example, the dosage form or pharmaceutical composition may comprise between about 1 mg and about 200 mg of the pharmaceutically active agent, e.g., between about 1 mg and about 100 mg, between about 1 mg and about 80 mg, between about 1 mg and about 70 mg, between about 1 mg and about 60 mg, between about 1 mg and about 50 mg, between about 1 mg and about 40 mg, between about 10 mg and about 40 mg, between about 10 mg and about 25 mg, between about 40 mg and about 60 mg, or between about 50 mg and about 70 mg, or between about 1 mg and about 10 mg, of the pharmaceutically active agent, e.g., about 3 mg, about 4 mg, about 5 mg, about 6 mg, about 8 mg, about 10 mg, about 12 mg, about 15 mg, about 20 mg, about 25 mg, about 30 mg, about 40 mg, about 50 mg, about 60 mg, or about 70 mg, of the pharmaceutically active agent. Also, for example, the dosage form or pharmaceutical composition may comprise between about 1 mg and about 400 mg of the pharmaceutically active agent, e.g., between about 1 mg and about 350 mg, between about 1 mg and about 300 mg, between about 1 mg and about 250 mg, between about 1 mg and about 200 mg, between about 10 mg and about 350 mg, between about 50 mg and about 300 mg, between about 75 mg and about 250 mg, between about 100 mg and about 200 mg, e.g., about 60 mg, about 70 mg, about 80 mg, about 100 mg, about 120 mg, about 140 mg, about 160 mg, about 180 mg, about 200 mg, about 220 mg, about 240 mg, about 260 mg, about 280 mg, or about 300 mg of the pharmaceutically active agent.

It will be understood that when the pharmaceutically active agent is a pharmaceutically acceptable salt of Formula (I), the amount of the pharmaceutically active agent in the dosage form or pharmaceutical composition will be slightly higher than the equivalent amount of free base. For example, a dosage form or pharmaceutical composition disclosed herein comprising 40 mg of pitolisant hydrochloride comprises about 35.6 mg of pitolisant (freebase). In some embodiments, a dosage form or pharmaceutical composition disclosed herein comprises about 40 mg of pitolisant monohydrochloride, or about 35.6 mg pitolisant (freebase). In some embodiments, a dosage form or pharmaceutical composition disclosed herein comprises about 20 mg of pitolisant monohydrochloride, or about 17.8 mg pitolisant (freebase).

The dosage form can comprise between about 1 mg and about 40 mg of an alkaline agent (e.g., magnesium carbonate, potassium bicarbonate, or sodium carbonate). For example, the dosage form can comprise about 1 mg, about 2 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg of an alkaline agent (sometimes referred to herein as a base). For example, the dosage form can comprise about 1 mg, about 2 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg of magnesium carbonate. For example, the dosage form can comprise about 1 mg, about 2 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg of potassium bicarbonate. For example, the dosage form can comprise about 1 mg, about 2 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg of sodium carbonate.

The dosage forms of the present disclosure can be tablets, caplets, capsules, suspensions, granules, powders, or the like. Preferably, the dosage forms of the present disclosure are delayed-release or enteric coated tablets, delayed-release or enteric coated caplets, delayed-release or enteric coated capsules, or the like. The tablet (e.g., delayed-release or enteric coated tablet) may be a round, biconvex tablet. The tablet may be engraved, e.g., with a number, letter(s), or both.

The dosage forms or pharmaceutical compositions of the present disclosure can further comprise one or more pharmaceutically acceptable excipients, such as diluents, dispersing agents, granulating agents, surface active agents, emulsifiers, disintegrating agents (sometimes referred to herein as disintegrants), binding agents (sometimes referred to herein as binders), preservatives, buffering agents, lubricating agents (sometimes referred to herein as lubricants), glidants, adjuvants, fillers, wetting agents, suspending agents, solvents, dispersion media, ion exchangers, salts, electrolytes, waxes, and/or oils, and the like.

Non-limiting examples of pharmaceutically acceptable excipients suitable for use in the dosage forms or pharmaceutical compositions disclosed herein include alumina, a phosphate (e.g., calcium phosphate, dicalcium phosphate, tricalcium phosphate, disodium hydrogen phosphate, potassium hydrogen phosphate), a sulfate (e.g., calcium sulfate), cellulose, cellulose derivatives, croscarmellose sodium, microcrystalline cellulose (including spray-dried microcrystalline cellulose), silicified microcrystalline cellulose, hydroxypropylmethylcellulose, methylcellulose, carboxymethylcellulose or salts thereof (e.g., sodium carboxymethylcellulose, or calcium carboxymethylcellulose), kaolin, bentonite, VEEGUM®, lactose (e.g., anhydrous lactose, spray dried lactose, hydrated lactose), mannitol, sorbitol, sucrose, inositol, compressible sugar, trehalose, xylitol, acacia, gelatin, glucose, maltodextrin, starch (e.g., corn starch, potato starch), sodium starch glycolate, starch derivatives, an amino acid (e.g., glycine or leucine), magnesium carbonate, polyvinylpyrrolidone (PVP, povidone) (e.g., crosslinked PVP, crospovidone), polyvinyl alcohol, tragacanth, polyethylene glycol, polymethacrylates, mineral clay powders, croscarmellose, poloxamer, fatty acids or salts thereof (e.g., lauric acid, sodium lauryl sulfate, stearic acid, calcium stearate, magnesium stearate, aluminum stearate, oleic acid), hydrogenated vegetable oils, talc, titanium dioxide, glyceryl behenate, silicon dioxide (e.g., colloidal silicon dioxide), anhydrous colloidal silica, a silicate salt (e.g., magnesium trisilicate), lecithin, serum protein (e.g., human serum albumin), sorbic acid, potassium sorbate, a metal cation salt (e.g., a sodium salt, such as sodium chloride, a potassium salt, such as potassium chloride, a magnesium salt, such as or magnesium chloride, a zinc salt, such as zinc chloride), water, dimethylacetamide, protamine sulfate, a polyacrylate, wool fat, ethylenediaminetetraacetic acid (EDTA), a cyclodextrin (e.g., CAPTISOL©), KOLLIDON® CL, CELLACTOSE®, LUDIPRESS®, polysorbates (e.g., TWEEN®, e.g., TWEEN® 20 or TWEEN® 80), and combinations thereof.

In a preferred aspect, a dosage form (e.g., oral dosage form) or pharmaceutical composition of the present disclosure comprises a combination of pharmaceutically acceptable excipients. For example, a dosage form or pharmaceutical composition of the present disclosure may comprise one or more, or all of, the following pharmaceutically acceptable excipients: lactose, microcrystalline cellulose (e.g., Vivapur 102), croscarmellose sodium (e.g., Vivasol GF LM), colloidal silica (e.g., aerosol 200 pharma), and magnesium stearate (e.g., Ligamed MF 2V). The pharmaceutically acceptable excipient may be present only in the core of the dosage form.

Each pharmaceutically acceptable excipient can be present in the dosage form or pharmaceutical composition in any suitable amount. For example, a pharmaceutically acceptable excipient can be present in the dosage form or pharmaceutical composition in an amount of between about 1% and about 95% by weight of the dosage form or pharmaceutical composition, e.g., between about 60% to 90%, or between about 80% to about 90%, by weight of the dosage form or pharmaceutical composition.

For example, the pitolisant may be present in the core of the dosage form in an amount of about 14 wt % to about 18 wt % (e.g., about 16 wt %), the alkaline agent (e.g., magnesium carbonate, potassium bicarbonate, or sodium carbonate) may be present in the core of the dosage form in an amount of about 10 wt % to about 14 wt % (e.g., about 12 wt %), and the pharmaceutically acceptable excipients combined may account for the remainder of the weight of the core of the dosage form. One pharmaceutically acceptable excipient may be a diluent (e.g., microcrystalline cellulose) that is present in the core of the dosage form in an amount of about 20% to about 30% by weight (e.g., about 21.4 wt %). Another pharmaceutically acceptable excipient may be a diluent (e.g., lactose) that is present in the core of the dosage form in an amount of about 40 wt % to about 50 wt % (e.g., about 42.9 wt %). Another pharmaceutically acceptable excipient may be a disintegrant (e.g., croscarmellose sodium) that is present in the core of the dosage form in an amount of about 3% to about 10% by weight (e.g., about 4.5 wt %). Another pharmaceutically acceptable excipient may be a lubricant (e.g., magnesium stearate) that is present in the core of the dosage form in an amount of about 1% to about 4% by weight (e.g., about 2.4 wt %). Another pharmaceutically acceptable excipient may be a flowing agent (e.g., colloidal silica) that is present in the core of the dosage form in an amount of about 0.1% to about 2% by weight (e.g., about 0.8 wt %).

Preferably, the dosage forms or pharmaceutical compositions of the present disclosure are administered orally.

Preparation of the dosage forms (e.g., oral dosage forms) or pharmaceutical composition of the present disclosure can include conventional methods, such as blending, filling, compressing (e.g., direct compression, compression of dry, wet or sintered granules), coating (e.g., coating in a spray process), extrusion, granulation (e.g., wet or dry granulation), pelleting (e.g., direct pelleting), binding, powder layering (e.g., onto active ingredient-free beads, or neutral cores or particles of pharmaceutically active agent), and rounding off.

For example, a dosage form of the present disclosure may be prepared with following steps: (a) preparing a core blend comprising pitolisant, an alkaline agent, and optionally one or more pharmaceutically acceptable excipients, e.g., by blending the pharmaceutically active agent (e.g., crystalline powder) with one or more pharmaceutically acceptable excipients (e.g., binder, disintegrant, glidant, etc.). Additional sieving steps may be employed. The next step may comprise (b) forming a core tablet with the blend, e.g., by tableting using a press. Optionally, the core tablet may be coated with an anti-moisture barrier coating disclosed herein, e.g., using a spray process. Finally, the core tablet, which may be coated with an anti-moisture barrier, can be further coated with a delayed-release (e.g., enteric) coating material disclosed herein, e.g., using a spray process. The coating steps may comprise spraying the anti-moisture barrier material or the delayed-release (e.g., enteric) coated material as a solution, suspension, or dispersion (e.g., an organic solution, or aqueous dispersion).

### Delayed-Release Coating

The dosage forms (e.g., oral dosage forms) and pharmaceutical compositions disclosed herein comprise a delayed-release (e.g., enteric) coating. The delayed-release (e.g., enteric) coating may be a material that is pH-dependent. For example, a dosage form or pharmaceutical composition disclosed herein may remain substantially intact in the stomach where the pH is low (about 1.0 to 3.5), and release the pharmaceutically active agent after entering the small intestine, due to disintegration of the enteric coating in the relatively higher pH of the small intestine (about 5.5 to 7.0). In some aspects of the present disclosure, the delayed-release (e.g., enteric) coating may disintegrate at a higher pH, e.g., to substantially release the pharmaceutically active agent in more distal portions of the small intestine where the pH is even higher (e.g., about 6.0 to 7.5). A pharmaceutical composition or dosage form of the present disclosure may substantially release the pharmaceutically active agent in the duodenum, in the jejunum, or in both the duodenum and jejunum.

The delayed-release (e.g., enteric) coating can be substantially resistant to disintegration in the stomach or gastric fluid, e.g., at a pH of between about 1.0 and about 3.5. The delayed-release (e.g., enteric) coating can disintegrate in the intestine (e.g., small intestine) to expose the pharmaceutically active agent, e.g., after the dosage form or pharmaceutical composition has entered the intestine. To that end, the delayed-release (e.g., enteric) coating may substantially disintegrate in the intestine (e.g., small intestine) or intestinal fluid, or at a pH of greater than 5 (e.g., a pH of greater than 5.5, greater than 6.0, greater than 6.5, greater than 7.0, or greater than 7.5).

A delayed-release (e.g., enteric) coating of a dosage form or pharmaceutical composition of the present disclosure may be acid resistant (gastric resistant) and prevent more than 10 percent of the pharmaceutically active agent to be released when the dosage form is in the stomach, in a hydrochloride solution (pH 1.2), in gastric fluid, or simulated gastric fluid, for at least 1 hour (e.g., 2 hours), e.g., releasing less than 10%, less than 8%, less than 6%, less than 4% less than 3%, less than 2%, less than 1% by weight, or none of the pharmaceutically active agent. The hydrochloride solution, gastric fluid, or simulated gastric fluid may be at a temperature of about 37° C. The dosage form or pharmaceutical composition, or the delayed-release (e.g., enteric) coating thereof, may substantially disintegrate after the dosage form or pharmaceutical composition is in the intestine (e.g., small intestine), or is exposed to intestinal fluid, simulated intestinal fluid, or a solution, e.g., buffer (e.g., phosphate buffer), with a pH of greater than 5 (e.g., a pH of greater than 5.5, 6.0, 6.5, 7.0, 7.5, or higher).

A dosage form or pharmaceutical composition according to the present disclosure may be a delayed release dosage form or pharmaceutical composition according to the United States Pharmacopeia (USP) 701 Procedure and Criteria for Delayed-Release Tablets and Capsules (tablets or capsules that are formulated with acid-resistant or enteric coatings).

A delayed-release (e.g., enteric) coating of a dosage form or pharmaceutical composition disclosed herein may comprise a polymer. For example, the polymer of the enteric coating may be a polymer comprising an ionizable functional group (e.g., a carboxylic acid group), e.g., a functional group that is not ionized in the stomach or gastric fluid, or in a solution with a pH of between about 1.0 and about 3.5. The functional group may be a group that is ionized in the intestine (e.g., small intestine) or intestinal fluid, or in a solution with a pH of greater than 5 (e.g., a pH of greater than 5.5, greater than 6.0, greater than 6.5, greater than 7.0, or greater than 7.5).

The delayed-release (e.g., enteric) coating may comprise an acrylic polymer, such as acrylic acid and methacrylic acid copolymers, methacrylate copolymers (e.g., copolymers of methyl methacrylate, butyl methacrylate and dimethylethyl methacrylate, copolymers of methyl methacrylate, ethyl acrylate and trimethylammoniumethyl methacrylate, copolymers of methyl methacrylate and ethyl acrylate); a cellulosic material, such as an alkylcellulose, e.g., ethylcellulose, AQUACOAT®, SURELEASE®, hydroxypropyl methylcellulose phthalate, hydroxypropyl methylcellulose acetate succinate, cellulose acetate trimellitate, cellulose acetate phthalate, or the like; polyvinylpyrrolidones (PVP), polyvinyl alcohols, polyvinyl alcohol-polyethylene glycol graft copolymers, a KOLLICOAT® polymer, starch, starch derivatives, polyvinyl acetate (PVAc), polyvinyl acetate phthalate (PVAP, or COATERIC®), a KOLLIDON® polymer, vinyl acetate-vinylpyrrolidone copolymer (e.g., KOLLIDON® VA64), vinyl acetate:crotonic acid copolymer (VAC:CRA), polyethylene glycols (e.g., polyethylene glycols with a molecular weight above 1000 g/mol), chitosan, crosslinked and/or non-crosslinked polyacrylic acid, sodium alginate, pectin, methacrylic acid ester copolymers, ethoxyethyl methacrylates, cyanoethyl methacrylate, poly(methacrylic acid), methacrylic acid alkylamide copolymer, poly(methyl methacrylate), polymethacrylates, poly(methyl methacrylate) copolymer, polyacrylamide, aminoalkyl methacrylate copolymer, poly(methacrylic acid anhydride), glycidyl methacrylate copolymers, ammonio methacrylate copolymers, or the like; shellac, zein, or the like; a combination thereof. In some embodiments, the delayed-release (e.g., enteric) coating comprises an anionic polymer, e.g., an anionic methacrylate copolymer. It will be understood that the polymer may not be anionic at sufficiently low pH (e.g., in gastric fluid) but becomes ionized upon increasing pH (e.g., in intestinal fluid).

The delayed-release (e.g., enteric) coating may comprise ACRYL-EZE®.

The delayed-release (e.g., enteric) coating may also comprise a plasticizer, a stabilizer (e.g., oleic acid, or a polysorbate e.g., TWEEN® 80), a lubricant, a glidant, a release agent, or a pigment (e.g., talc, titanium dioxide, or magnesium stearate). Examples of suitable plasticizers include sebacates (e.g., dibutyl sebacate), propylene glycol, polyethylene glycols (e.g., PEG8000), phthalates (e.g., diethyl phthalate or dibutyl phthalate) or phthalate esters, citric acid esters (e.g., triethyl citrate, tibutyl citrate), triacetin, acetylated monoglycerides, castor oil, propylene glycol, and polyethylene glycols. Examples of release agents include glycerol monostearate or other suitable fatty acid derivatives, silica derivatives, and talc. Preferred stabilizers are PEG8000 and triethyl citrate.

The delayed-release (e.g., enteric) coating may comprise a material, or may be a delayed-release (e.g., enteric coating), disclosed in US 2004/0028737, US 2005/0271778, WO 2005/044240, WO 2007/006353, WO 2008/135090, each of which are incorporated herein by reference in their entireties.

### Anti-Moisture Barrier

The dosage forms (e.g., oral dosage forms) and pharmaceutical compositions disclosed herein may comprise an anti-moisture barrier coating. The anti-moisture barrier is distinct from the delayed-release (e.g., enteric coating). The anti-moisture barrier coating may be positioned around the core of a dose form and underneath the delayed-release (e.g., enteric) coating layer (i.e., between the outer surface of the core and the inner surface of the delayed-release (e.g., enteric) coating layer). The anti-moisture barrier may comprise any suitable material, such as material that prevents moisture from the environment readily entering the core of the dose form prior to its intended dissolution (e.g., upon administration).

The anti-moisture barrier may comprise a polymer. Suitable polymers include polyvinyl alcohols and hypromellose (hydroxypropylmethylcellulose). An exemplary anti-moisture barrier material of the present disclosure is OPADRY® amb II.

### Pharmaceutically Active Agent

The pharmaceutically active agent of the dosage form (e.g., oral dosage form) or pharmaceutical composition disclosed herein is pitolisant (1-[3-[3-(4-chlorophenyl)propoxy]propyl]-piperidine), or a pharmaceutically acceptable salt, solvate, or hydrate thereof. Pitolisant is represented by Formula (I):

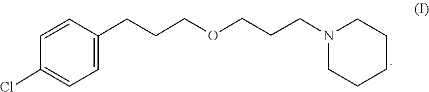 [SMILES: Clc1ccc(CCCOCCCN2CCCCC2)cc1]

The pharmaceutically active agent may be Formula (I) in freebase form, or may be any pharmaceutically acceptable salt of Formula (I), e.g., a pharmaceutically acceptable salt disclosed herein. For example, the pharmaceutically active agent can be a hydrochloride salt or an oxalate salt of Formula (I).

In preferred aspects, the pharmaceutically active agent is a hydrochloride salt of Formula (I), e.g., (1-[3-[3-(4-chlorophenyl)propoxy]propyl]-piperidine monohydrochloride; pitolisant monohydrochloride), represented by Formula (Ia):

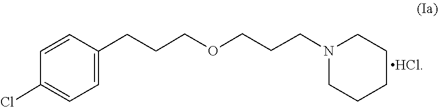 [SMILES: Clc1ccc(CCCOCCCN2CCCCC2)cc1]

Without wishing to be bound by theory, it is believed that pitolisant can activate receptors within the stomach, such as histamine H₃ receptors, which may cause gastric side effects associated with existing pitolisant formulations such as WAKIX®. The inventors discovered that by formulating pitolisant dosage forms or pharmaceutical compositions with an enteric coating, the gastric side effects can be substantially reduced or eliminated. Without wishing to be bound by theory, it is also believed that formulating pitolisant with an alkaline agent can improve Cₘₐₓ or Tₘₐₓ of pitolisant, relative to comparative formulations such as WAKIX®, and the overall exposure (as measured by AUC) is better between the dosage forms of the present disclosure and WAKIX®. In other words, despite their enteric coatings, the dosage forms and pharmaceutical compositions disclosed herein may be substantially bioequivalent to, or have improved bioavailability, as compared to WAKIX®.

The dosage forms and pharmaceutical compositions disclosed herein may comprise the monohydrochloride salt of Formula (I), which has a relatively high aqueous solubility, e.g., compared to other known pharmaceutically acceptable salts of Formula (I) (e.g., pitolisant oxalate). The aqueous solubility of the pharmaceutically active agent may be about 4 g/mL at 23° C.

The pharmaceutically active agent (e.g., Formula (I), or Formula (Ia)) may be crystalline.

The pharmaceutically active agent can have a water content of 6% (±0.5%) or less, e.g., less than 5%, less than 4%, less than 3%, less than 2%, or less than 1%, by weight.

The pharmaceutically active agent can have characteristic IR peaks at 1112, 1101, 2936, 2868, 1455, 2647, 2551, 1492, and 802 cm⁻¹ (±5).

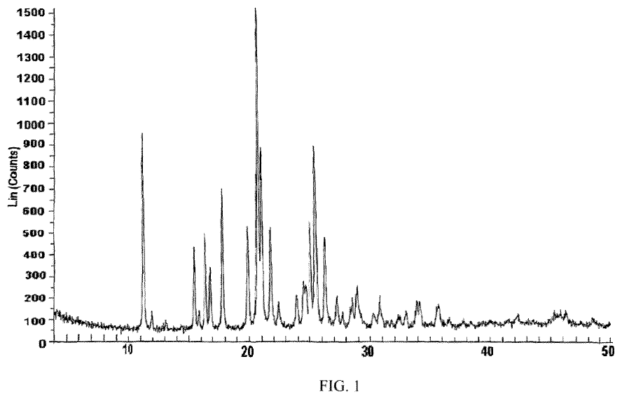

The pharmaceutically active agent can have an X-ray powder diffraction pattern with characteristic peaks (2θ): 11.2°, 19.9°, 20.7° and 34.1° (±0.2°), as obtained using X-Ray diffraction technique described in U.S. Pat. No. 8,207,197, using a Nonius Kappa Charge Coupled Device system at −158° C., operating at a wavelength of 0.71073 Å. The pharmaceutically active agent can have an X-ray powder diffraction pattern as shown in FIG. 1.

The pharmaceutically active agent may be a compound disclosed in U.S. Pat. Nos. 8,207,197, 8,354,430, 8,486,947, 7,138,413, 7,910,605, or U.S. Pat. No. 7,169,928, each of which are incorporated herein by reference in their entireties.

### Pharmacokinetics

Without wishing to be bound by theory, it is believed that the oral dosage forms and pharmaceutical compositions disclosed herein are substantially bioequivalent, or have improved bioavailability, relative to their non-enterically coated counterparts that do not have an alkaline agent in the core, such as WAKIX®. In other words, and without wishing to be bound by theory, it is believed that the presence of the alkaline agent in the core and/or the delayed-release (e.g., enteric) coating of the dosage forms or pharmaceutical compositions disclosed herein either does not negatively impact, or improves their pharmacokinetics and/or bioavailability following administration, resulting in a superior biological effect, bioavailability, and/or pharmacokinetics as would be expected by administering an equivalent dosage form or pharmaceutical composition without an enteric coating or alkaline agent (e.g., otherwise containing the same pharmaceutically active agent and pharmaceutically acceptable excipient(s), in substantially the same amounts), such as WAKIX®.

In some embodiments, an oral dosage form or pharmaceutical composition disclosed herein is bioequivalent to WAKIX®, as determined by the Bioequivalence Guidance 94D-0401 provided by the U.S. Food and Drug Administration (FDA).

In some embodiments, an oral dosage form or pharmaceutical composition disclosed herein has improved bioavailability relative to WAKIX®. For example, an oral dosage form or pharmaceutical composition disclosed herein may provide a bioavailability of pitolisant that is at least 1% greater than WAKIX® when administered in substantially the same manner (e.g., by oral administration) and/or in substantially the same amount. For example, an oral dosage form or pharmaceutical composition disclosed herein may provide a relative increase in bioavailability of pitolisant of between about 1% and about 100%, e.g., between about 2% and about 50%, between about 5% and about 50%, between about 10% and about 50%, between about 15% and about 50%, e.g., about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 50%, about 60%, about 70%, about 80%, about 90%, or about 100%, or more, relative to WAKIX®. In some embodiments, an oral dosage form or pharmaceutical composition disclosed herein provides a relative increase in bioavailability of pitolisant of about 20%, or more, relative to WAKIX®.

### Methods of Treatment

The present disclosure further relates to a method for the treatment of a disease or disorder, comprising administering a dosage form (e.g., oral dosage form) or pharmaceutical composition disclosed herein to a subject in need thereof. The disease or disorder may be fatigue, a sleep disorder (e.g., excessive daytime sleepiness (EDS), cataplexy, narcolepsy, sleep apnea (e.g., obstructive sleep apnea), sleep induced apnea, diurnal somnolence, idiopathic hypersomnia (IH), sleep inertia (e.g., sleep inertia in IH), central nervous system disorder (e.g., epilepsy, Alzheimer's disease, Parkinson's disease, dementia (e.g., dementia with Lewy bodies and/or vascular dementia), attention disorders, wakefulness disorders, memorization disorders, cognitive deficits (e.g., in aged persons), psychiatric pathologies, depressive and asthenic states, vertigo, and motion sickness, obesity, psychosomatic disorders, respiratory disorders, allergic conditions, inflammatory conditions, cardiac conditions, gastrointestinal conditions, conditions of the urogenital system, conditions of the cutaneous system, stress, migraine, headache, pain, psychotropic disorders, asthma, bronchitis, rhinitis, tracheitis, gastric ulcers, duodenal ulcers, ulcerative colitis, Crohn's disease, irritable bowel syndrome, cystitis, metritis, urinary incontinence, fecal incontinence, urticaria, itching, arthritis, conjunctivitis, premenstrual syndrome, prostatic inflammations, genital disorders, rheumatic conditions, ocular conditions, sialorrhea, convulsion, depression, disorders of the hypothalamo-hypophyseal system, disorders of the cerebral circulation, and disorders of the immune system.

In preferred aspects, the disease or disorder is a sleep disorder. For example, the disease or disorder can be excessive daytime sleepiness (EDS). The EDS can be in subjects (e.g., adult subjects) with narcolepsy.

The present disclosure further relates to a method for the prevention of undesirable side effects associated with using antipsychotic or antidepressant agents (e.g., aripiprazole, clozapine, olanzapine, risperidone, quetiapine, sertindole, mirtazapine, amitryptiline, and paroxetine), comprising administering a dosage form or pharmaceutical composition of the present disclosure to a subject in need thereof. Non-limiting examples of undesirable side effects associated with using antipsychotic or antidepressant agents includes weight gain, somnolence, and cognitive impairment.

The present disclosure further relates to a method for (i) inducing an extended state of wakefulness; (ii) improving cognitive processes; (iii) reducing food intake; and/or (iv) normalizing vestibular reflexes, comprising administering a dosage form or pharmaceutical composition disclosed herein to a subject in need thereof.

A dosage form (e.g., oral dosage form) or pharmaceutical composition disclosed herein may be taken once daily, twice daily, or more often. More than one dosage form can be administered at once to achieve a desired dose. Preferably, the dosage form (or dosage forms, when multiple dosage forms are used) or pharmaceutical composition is/are taken once daily.

For example, one or more dosage forms disclosed herein may be administered orally once daily, e.g., in the morning upon wakening.

One or more dosage forms or pharmaceutical compositions disclosed herein may be taken with a dosing frequency (e.g., once daily) so that the total amount of pharmaceutically active agent administered is within the range of from about 10 mg to about 400 mg per day, e.g., about 15 mg to about 300 mg per day, about 15 mg to about 250 mg per day, between about 15 mg to about 200 mg per day, between about 30 mg to about 250 mg per day, between about 40 mg to about 250 mg per day, between about 80 mg to about 250 mg per day, between about 100 mg to about 250 mg per day, e.g., about 20 mg per day, about 30 mg per day, about 40 mg per day, about 50 mg per day, about 60 mg per day, about 70 mg per day, about 80 mg per day, about 90 mg per day, about 100 mg per day, about 110 mg per day, about 120 mg per day, about 130 mg per day, about 140 mg per day, about 150 mg per day, about 160 mg per day, about 170 mg per day, about 180 mg per day, about 190 mg per day, about 200 mg per day, about 210 mg per day, about 220 mg per day, about 230 mg per day, about 240 mg per day, about 250 mg per day, about 260 mg per day, or more. One or more dosage forms or pharmaceutical compositions disclosed herein may be taken with a frequency (e.g., once daily) so that the total amount of pharmaceutically active agent administered is within the range of from about 10 mg to about 100 mg per day, e.g., about 20 mg to about 80 mg per day. One or more dosage forms or pharmaceutical compositions disclosed herein may be taken with a frequency (e.g., once daily) so that the total amount of pharmaceutically active agent administered is within the range of from about 10 mg to about 50 mg per day, e.g., about 15 mg to about 40 mg per day. One or more dosage forms or pharmaceutical compositions disclosed herein may be taken with a frequency (e.g., once daily) so that the total amount of pitolisant (in terms of freebase) administered is within the range of from about 17.8 mg to about 35.6 mg per day. A subject may be administered orally one dosage form once daily, where each dosage form comprises 35.6 mg pitolisant (in terms of freebase), to achieve a daily dose of 35.6 mg pitolisant (free base). A subject may be administered orally one or more dosage forms on a daily basis (e.g., once daily), to achieve a total daily dose of 180 mg per day.

### EXAMPLES

In order that the invention described herein may be more fully understood, the following examples are set forth. These examples are offered to illustrate the dose forms (e.g., delayed-release (e.g., enteric-coated) dose forms), methods of use, and methods of making, and are not to be construed in any way as limiting their scope.

The compounds and dose forms provided herein can be prepared from readily available starting materials using modifications to the specific protocols set forth below that would be well known to those of ordinary skill in the art. It will be appreciated that where typical or preferred process conditions are given, other process conditions can also be used unless otherwise stated. Optimum reaction conditions may vary with the particular reactants or solvents used, but such conditions can be determined by those skilled in the art by routine optimization.

### Materials and Methods

Materials used in the examples set forth below were purchased from commercial suppliers. Pitolisant hydrochloride (API) was obtained from Corden Pharma Chenôve (MA-22-061). Pitolisant hydrochloride may also be prepared according to the methods described in U.S. Pat. No. 8,207,197, which is incorporated herein by reference in its entirety. Microcrystalline cellulose (Vivapur 102) (diluent) was obtained from JRS Pharma (ME-23-057). Magnesium carbonate (alkaline agent) was obtained from Paul Lohmann (ME-22-070). Potassium bicarbonate (Hydrogenocarbonate De Potassium EMPROVE® ESSENTIAL) (alkaline agent) was obtained from Merck (ME-23-048). Sodium carbonate (Sodium Carbonate Anhydrous EMPROVE® ESSENTIAL) (alkaline agent) was obtained from Merck (ME-23-047). Anhydrous lactose (Supertab 24AN) was obtained from DFE (ME-23-051). Croscarmellose sodium (Vivasol GF LM) (disintegrant) was obtained from JRS Pharma (ME-22-081). Magnesium stearate (Ligamed MF 2V) (lubricant) was obtained from Peter Greven (ME-20-033). Anhydrous colloidal silica (Aerosil 200 Pharma) (flowing agent) was obtained from Evonik (ME-23-056). OPADRY® amb II clear (anti-moisture barrier agent) was obtained from Colorcon (ME-22-082). ACRYL-EZE® Clear (gastro resistant coating agent) was also obtained from Colorcon (ME-22-083). Water for injection (solvent) was obtained from Lavoisier (ME-22-056) (purified water can also be used). PEG8000 (plasticizer) was obtained from Clariant (ME-22-073).

### Example 1. Preparation of Exemplary Pitolisant Core Tablets (Batch 1 Core)

### Manufacturing Formula

Clinical manufacturing of gastro resistant (GR) film-coated tablets was carried out for the supply of a phase I clinical study (NF1-BF2.649/P22-06) to assess relative bioavailability compared to WAKIX® 18 mg, film-coated immediate release tablets.

Four batches of pitolisant hydrochloride 40 mg gastro resistant film-coated tablets (i.e., tablets with a delayed release coating) were manufactured with different formulations:

One batch with magnesium carbonate 30 mg/tablet as alkaline buffer (formulation 1)

One batch with magnesium carbonate 7.5 mg/tablet as alkaline buffer (formulation 2)

One batch with potassium bicarbonate 30 mg/tablet as alkaline buffer (formulation 3)

One batch with sodium carbonate 30 mg/tablet as alkaline buffer (formulation 4)

Part of these active batches were used for a stability study and sampled by Synerlab Developpement QC (see Example 2).

Film-coating was performed in one sub-lot using the Manesty coating pan (330 mm pan) in two steps: first step corresponding to the first layer (clear ambII film-coating), then second step corresponding to the gastro-resistant (GR) layer (clear acryl eze film coating).

Tablets were prepared using the manufacturing formula described in Tables 1-4.

TABLE 1
Active Batch - Formulation 1.

| | | Unit | Centesimal formula (%) | | Manufacturing |
|---|---|---|---|---|---|
| Raw material | Function | formula (mg) | Core | Coated | formula (g) |
| Pitolisant HCl | API | 40.00 | 16.00 | 13.37 | 240.00* |
| Magnesium | Alkaline | 30.00 | 12.00 | 10.03 | 180.00 |
| carbonate | buffer | | | | |
| Lactose | Filler | 107.17 | 42.87 | 35.82 | 643.02 |
| (Supertab 24AN) | | | | | |
| Microcrystalline | Filler | 53.58 | 21.43 | 17.91 | 321.48 |
| cellulose | | | | | |
| (Vivapur 102) | | | | | |
| Croscarmellose | Disintegrant | 11.25 | 4.50 | 3.76 | 67.50 |
| sodium (Vivasol | | | | | |
| GF LM) | | | | | |
| Colloidal silica | Anti-tacking | 2.00 | 0.80 | 0.67 | 12.00 |
| (aerosol 200 | agent | | | | |
| pharma) | | | | | |
| Magnesium | Lubricant | 6.00 | 2.40 | 2.01 | 36.00 |
| stearate | | | | | |
| (Ligamed MF-2V) | | | | | |
| TOTAL | | 250.00 | 100.0 | 83.55 | 1500.00 |
| 1^{st} COATING | | | | | |
| Core tablet | Core tablet | 250.0 | / | 83.55 | 1000.00 |
| Opadry amb II¹ | Coating | 7.50 | / | 2.51 | 75.00 |
| (Clear, 88A190022) | (base) | | | | |
| Water for | Solvent | / | / | / | 425.00 |
| injection⁴ | | | | | |
| 2^{nd} COATING | | | | / | |
| Acryl eze² | Gastro | 38.625 | / | 12.91 | 300.00 |
| 93A19346 clear | resistant | | | | |
| | coating | | | | |
| PEG 8000³ | Plasticizer | 3.09 | | 1.03 | 24.00 |
| Water for | Solvent | / | / | / | 1200.00 |
| injection⁴ | | | | | |
| TOTAL | | 299.215 | / | 100.0 | / |

¹15% suspension prepared in excess (x2.5), weight gain = 3%
²20% suspension prepared in excess (x2.0), weight gain = 15%
³PEG at 8% of acryl eze
⁴Evaporated during process
*for readjustment, pitolisant HCl assay and water content for API batch was taken into account

TABLE 2
Active Batch - Formulation 2

| | | Unit | Centesimal formula (%) | | Manufacturing |
|---|---|---|---|---|---|
| Raw material | Function | formula (mg) | Core | Coated | formula (g) |
| Pitolisant HCl | API | 40.00 | 16.00 | 13.37 | 240.00* |
| Magnesium | Alkaline | 7.50 | 3.00 | 2.51 | 45.00 |
| carbonate | buffer | | | | |
| Lactose | Filler | 122.17 | 48.87 | 40.83 | 733.02 |
| (Supertab 24AN) | | | | | |
| Microcrystalline | Filler | 61.08 | 24.43 | 20.41 | 366.48 |
| cellulose | | | | | |
| (Vivapur 102) | | | | | |
| Croscarmellose | Disintegrant | 11.25 | 4.50 | 3.76 | 67.50 |
| sodium (Vivasol | | | | | |
| GF LM) | | | | | |
| Colloidal silica | Anti-tacking | 2.00 | 0.80 | 0.67 | 12.00 |
| (aerosol 200 | agent | | | | |
| pharma) | | | | | |
| Magnesium | Lubricant | 6.00 | 2.40 | 2.01 | 36.00 |
| stearate | | | | | |
| (Ligamed MF-2V) | | | | | |
| TOTAL | | 250.00 | 100.0 | 83.55 | 1500.00 |
| 1^{st} COATING | | | | | |
| Core tablet | Core tablet | 250.0 | / | 83.55 | 1000.00 |
| Opadry amb II¹ | Coating | 7.50 | / | 2.51 | 75.00 |
| (Clear, 88A190022) | (base) | | | | |
| Water for | Solvent | / | / | / | 425.00 |
| injection⁴ | | | | | |
| 2^{nd} COATING | | | | / | |
| Acryl eze² | Gastro | 38.625 | / | 12.91 | 300.00 |
| 93A19346 clear | resistant | | | | |
| | coating | | | | |
| PEG 8000³ | Plasticizer | 3.09 | | 1.03 | 24.00 |
| Water for | Solvent | / | / | / | 1200.00 |
| injection⁴ | | | | | |
| TOTAL | | 299.215 | / | 100.0 | / |

¹15% suspension prepared in excess (x2.5), weight gain = 3%
²20% suspension prepared in excess (x2.0), weight gain = 15%
³PEG at 8% of acryl eze
⁴Evaporated during process
*for readjustment, pitolisant HCl assay and water content for API batch was taken into account

TABLE 3
Active Batch - Formulation 3

| | | | | | Manufac- |
|---|---|---|---|---|---|
| | | Unit | Centesimal | | turing |
| | | formula | formula (%) | | formula |
| Raw material | Function | (mg) | Core | Coated | (g) |
| Pitolisant HCl | API | 40.00 | 16.00 | 13.37 | 240.00* |
| Potassium | Alkaline | 30.00 | 12.00 | 10.03 | 180.00 |
| bicarbonate | buffer | | | | |
| Lactose | Filler | 107.17 | 42.87 | 35.82 | 643.02 |
| (Supertab | | | | | |
| 24AN) | | | | | |
| Microcrystalline | Filler | 53.58 | 21.43 | 17.91 | 321.48 |
| cellulose | | | | | |
| (Vivapur 102) | | | | | |
| Croscarmellose | Disintegrant | 11.25 | 4.50 | 3.76 | 67.50 |
| sodium | | | | | |
| (Vivasol GF | | | | | |
| LM) | | | | | |
| Colloidal silica | Anti-tacking | 2.00 | 0.80 | 0.67 | 12.00 |
| (aerosol 200 | agent | | | | |
| pharma) | | | | | |
| Magnesium | Lubricant | 6.00 | 2.40 | 2.01 | 36.00 |
| stearate | | | | | |
| (Ligamed MF- | | | | | |
| 2V) | | | | | |
| TOTAL | | 250.00 | 100.0 | 83.55 | 1500.00 |
| 1^{st} COATING | | | | | |
| Core tablet | Core tablet | 250.0 | / | 83.55 | 1000.00 |
| Opadry amb II¹ | Coating | 7.50 | / | 2.51 | 75.00 |
| (Clear, | (base) | | | | |
| 88A190022) | | | | | |
| Water for | Solvent | / | / | / | 425.00 |
| injection⁴ | | | | | |
| 2^{nd} COATING | | | | / | |
| Acryl eze² | Gastro | 38.625 | / | 12.91 | 300.00 |
| 93A19346 clear | resistant | | | | |
| | coating | | | | |
| PEG 8000³ | Plasticizer | 3.09 | | 1.03 | 24.00 |
| Water for | Solvent | / | / | / | 1200.00 |
| injection⁴ | | | | | |
| TOTAL | | 299.215 | / | 100.0 | / |

¹15% suspension prepared in excess (x2.5), weight gain = 3%
²20% suspension prepared in excess (x2.0), weight gain = 15%
³PEG at 8% of acryl eze
⁴Evaporated during process
*for readjustment, pitolisant HCl assay and water content for API batch was taken into account

TABLE 4
Active Batch - Formulation 4

| | | | | | Manufac- |
|---|---|---|---|---|---|
| | | Unit | Centesimal | | turing |
| | | formula | formula (%) | | formula |
| Raw material | Function | (mg) | Core | Coated | (g) |
| Pitolisant HCl | API | 40.00 | 16.00 | 13.37 | 240.00* |
| Sodium | Alkaline | 30.00 | 12.00 | 10.03 | 180.00 |
| carbonate | buffer | | | | |
| Lactose | Filler | 107.17 | 42.87 | 35.82 | 643.02 |
| (Supertab | | | | | |
| 24AN) | | | | | |
| Microcrystalline | Filler | 53.58 | 21.43 | 17.91 | 321.48 |
| cellulose | | | | | |
| (Vivapur 102) | | | | | |
| Croscarmellose | Disintegrant | 11.25 | 4.50 | 3.76 | 67.50 |
| sodium | | | | | |
| (Vivasol GF | | | | | |
| LM) | | | | | |
| Colloidal silica | Anti-tacking | 2.00 | 0.80 | 0.67 | 12.00 |
| (aerosol 200 | agent | | | | |
| pharma) | | | | | |
| Magnesium | Lubricant | 6.00 | 2.40 | 2.01 | 36.00 |
| stearate | | | | | |
| (Ligamed MF- | | | | | |
| 2V) | | | | | |
| TOTAL | | 250.00 | 100.0 | 83.55 | 1500.00 |
| 1^{st} COATING | | | | | |
| Core tablet | Core tablet | 250.0 | / | 83.55 | 1000.00 |
| Opadry amb II¹ | Coating | 7.50 | / | 2.51 | 75.00 |
| (Clear, | (base) | | | | |
| 88A190022) | | | | | |
| Water for | Solvent | / | / | / | 425.00 |
| injection⁴ | | | | | |
| 2^{nd} COATING | | | | / | |
| Acryl eze² | Gastro | 38.625 | / | 12.91 | 300.00 |
| 93A19346 clear | resistant | | | | |
| | coating | | | | |
| PEG 8000³ | Plasticizer | 3.09 | | 1.03 | 24.00 |
| Water for | Solvent | / | / | / | 1200.00 |
| injection⁴ | | | | | |
| TOTAL | | 299.215 | / | 100.0 | |

¹15% suspension prepared in excess (x2.5), weight gain = 3%
²20% suspension prepared in excess (x2.0), weight gain = 15%
³PEG at 8% of acryl eze
⁴Evaporated during process
*for readjustment, pitolisant HCl assay and water content for API batch was taken into account

### Blending Step

Prior to tableting, the manufacturing formulae were subjected to sieving and blending steps. API was sieved in sandwich on 800 μm with cellulose, silica, and alkalinizing agent (magnesium carbonate, potassium bicarbonate, or sodium carbonate) and mixed for 100 rotations. Lactose and croscarmellose was sieved on 800 μm and added to first blend before mixing for 100 rotations. Then, lubrication was performed with magnesium stearate for 50 rotations. The lubricated blend was tableted with tableting machine Styl'One evolution.

### Tableting Step

D8R16 punches were used for the tableting step. Mass of tablets was targeted at 250 mg. Compression force was set in order to reach a disintegration time below 15 minutes (target around 4 minutes) and a friability compliant with coating (<0.3, target <0.1%), hardness values will be collected for information as well (target around 70N). Bulk tablets were stored in double LDPE bags with desiccants and PP bucket at 9-25° C. prior to the film-coating step.

### Film-Coating Step

Tablet was used for coating (1.0 kg). Tablets were film-coated in two steps: first coating with clear Opadry (clear Amb II), weight gain at 3%, and second coating with acryl eze, weight gain at 15% (15% of gastro resistant, i.e., 16.2% weight gain total). The pan of the coating machine being suitable for a batch size of 1.0 kg, one sub-lot was suitable.

### Example 2. Stability of 40 mg Gastro-Resistant Film-Coated Tablets

Gastro resistant film-coated tablets containing an alkaline agent were developed to create a micro alkaline environment around the pitolisant HCl during its release in situ, that could enhance the absorption of the active (which was in its non-ionized form, based on its pKa of approximately 10.5).

Two manufacturing processes (direct compression and wet granulation), two alkaline agents (magnesium carbonate [associated with docusate in wet granulation process] and meglumine) and two forms of the pitolisant HCl (original and micronized) were compared.

Data was obtained after six months of storage at long term storage conditions, 25° C./60% RH and at accelerated storage conditions, 40° C./75% RH.

The specifications for each formulation are provided in Tables 5-10.

TABLE 5
Pitolisant HCl formulation for 40 mg gastro-resistant
film-coated tablets (LF22133-1) - wet granulation

| | | Unit | Centesimal | Manufacturing |
|---|---|---|---|---|
| | | formula | formula | formula |
| Raw material | Function | (mg) | (%) | (g) |
| Internal phase | | | | |
| Pitolisant HCl | API | 40.00 | 16.02 | 144.3 |
| Magnesium | Alkaline | 30.00 | 12.00 | 108.049 |
| carbonate | buffer | | | |
| Croscarmellose | Disintegrant | 3.75 | 1.50 | 13.488 |
| sodium | | | | |
| Povidone | Binder | 7.50 | 3.00 | 27.020 |
| Cellulose | Filler | 34.83 | 13.92 | 125.406 |
| microcrystalline | | | | |
| Docusate | Surfactant | 0.25 | 0.10 | 0.9035 |
| sodium | | | | |
| Purified water | Solvent | / | / | 20.009 |
| External phase | | | | |
| Granules | / | 116.33 | (46.54) | 391.2 |
| Cellulose | Filler | 17.42 | 6.97 | 58.570 |
| microcrystalline | | | | |
| Tribasic | Filler | 104.50 | 41.79 | 351.3 |
| calcium | | | | |
| phosphate | | | | |
| Croscarmellose | Disintegrant | 3.75 | 1.50 | 12.603 |
| sodium | | | | |
| Silica colloidal | Glidant | 2.00 | 0.80 | 6.726 |
| anhydrous | | | | |
| Magnesium | Lubricant | 6.00 | 2.40 | 20.182 |
| stearate | | | | |
| TOTAL | | 250.00 | 100.0 | 840.581 |
| Film-coating | | | | |
| Opadry amb II | Coating | 2.50 | | |
| 88A190022 | agent | | | |
| clear | | | | |

TABLE 6
Pitolisant HCl formulation for 40 mg gastro-resistant
film-coated tablets (LF22134-1_- wet granulation

| | | Unit | Centesimal | Manufacturing |
|---|---|---|---|---|
| | | formula | formula | formula |
| Raw material | Function | (mg) | (%) | (g) |
| Internal phase | | | | |
| Pitolisant HCl | API | 40.00 | 16.02 | 112.1 |
| Magnesium | Alkaline | 30.00 | 12.00 | 84.017 |
| carbonate | buffer | | | |
| Croscarmellose | Disintegrant | 3.75 | 1.50 | 10.498 |
| sodium | | | | |
| Povidone | Binder | 7.50 | 3.00 | 21.001 |
| Cellulose | Filler | 34.83 | 13.92 | 97.515 |
| microcrystalline | | | | |
| Docusate | Surfactant | 0.25 | 0.10 | 0.7010 |
| sodium | | | | |
| Purified water | Solvent | / | / | 15.999 |
| External phase | | | | |
| Granules | / | 116.33 | (46.54) | 306.5 |
| Cellulose | Filler | 17.42 | 6.97 | 45.891 |
| microcrystalline | | | | |
| Tribasic | Filler | 104.50 | 41.79 | 275.4 |
| calcium | | | | |
| phosphate | | | | |
| Croscarmellose | Disintegrant | 3.75 | 1.50 | 9.891 |
| sodium | | | | |
| Silica colloidal | Glidant | 2.00 | 0.80 | 5.271 |
| anhydrous | | | | |
| Magnesium | Lubricant | 6.00 | 2.40 | 15.208 |
| stearate | | | | |
| TOTAL | | 250.00 | 100.0 | 658.161 |
| Film-coating | | | | |
| Opadry amb II | Coating | 2.50 | | |
| 88A190022 | agent | | | |
| clear | | | | |
| ACRYL-EZE | Coating | 25.00 | | |
| 93A19346 clear | agent | | | |
| PEG8000 | Plasticizer | 2.0 | | |
| TOTAL | / | 279.5 | | / |

TABLE 7
Pitolisant HCl formulation for 40 mg gastro-resistant
coated tablets (LF22135-1) - direct compression

| | | Unit | Centesimal | Manufacturing |
|---|---|---|---|---|
| | | formula | formula | formula |
| Raw material | Function | (mg) | (%) | (g) |
| Internal phase | | | | |
| Pitolisant HCl | API | 40.00 | 14.31 | 144.2 |
| Meglumine | Alkaline | 23.74 | 8.49 | 85.462 |
| | buffer | | | |
| Cellulose | Filler | 55.67 | 19.92 | 200.4 |
| microcrystalline | | | | |
| Tribasic | Filler | 111.34 | 39.84 | 401.2 |
| calcium | | | | |
| phosphate | | | | |
| Croscarmellose | Disintegrant | 11.25 | 4.03 | 40.491 |
| sodium | | | | |
| Silica colloidal | Glidant | 2.00 | 0.72 | 7.192 |
| anhydrous | | | | |
| Magnesium | Lubricant | 6.00 | 2.15 | 21.604 |
| stearate | | | | |
| TOTAL | | 250.00 | (89.45) | 900.549 |
| Film-coating | | | | |
| Opadry amb II | Coating | 2.50 | 0.89 | |
| 88A190022 | agent | | | |
| clear | | | | |
| ACRYL-EZE | Coating | 25.00 | 8.94 | |
| 93A19346 clear | agent | | | |
| PEG8000 | Plasticizer | 2.0 | 0.72 | |
| TOTAL | / | 279.5 | 100 | / |

TABLE 8
Pitolisant HCl formulation for 40 mg gastro-resistant
coated tablets (LF22136-1_- direct compression

| | | Unit | Centesimal | Manufacturing |
|---|---|---|---|---|
| | | formula | formula | formula |
| Raw material | Function | (mg) | (%) | (g) |
| Internal phase | | | | |
| Pitolisant HCl | API | 40.00 | 14.31 | 111.9 |
| (micronized) | | | | |
| Meglumine | Alkaline | 23.74 | 8.49 | 66.4 |
| | buffer | | | |
| Cellulose | Filler | 54.34 | 19.44 | 152.2 |
| microcrystalline | | | | |
| Tribasic | Filler | 108.67 | 38.88 | 304.2 |
| calcium | | | | |
| phosphate | | | | |
| Croscarmellose | Disintegrant | 11.25 | 4.03 | 31.574 |
| sodium | | | | |
| Silica colloidal | Glidant | 3.00 | 1.07 | 8.401 |
| anhydrous | | | | |
| Magnesium | Lubricant | 9.00 | 3.22 | 25.200 |
| stearate | | | | |
| TOTAL | | 250.00 | (89.45) | (699.875) |
| Film-coating | | | | |
| Opadry amb II | Coating | 2.50 | 0.89 | |
| 88A190022 | agent | | | |
| clear | | | | |
| ACRYL-EZE | Coating | 25.0 | 8.94 | |
| 93A19346 clear | agent | | | |
| PEG8000 | Plasticizer | 2.0 | 0.72 | |
| TOTAL | / | 279.5 | 100 | / |

TABLE 9
Pitolisant HCl formulation for 40 mg gastro-resistant
coated tablets (LF22137-1) - direct compression

| | | Unit | Centesimal | Manufacturing |
|---|---|---|---|---|
| | | formula | formula | formula |
| Raw material | Function | (mg) | (%) | (g) |
| Internal phase | | | | |
| Pitolisant HCl | API | 40.00 | 14.31 | 144.0 |
| Magnesium | Alkaline | 30.00 | 10.73 | 108.1 |
| carbonate | buffer | | | |
| Cellulose | Filler | 53.58 | 19.17 | 192.9 |
| microcrystalline | | | | |
| Tribasic | Filler | 107.17 | 38.34 | 385.9 |
| calcium | | | | |
| phosphate | | | | |
| Croscarmellose | Disintegrant | 11.25 | 4.03 | 40.491 |
| sodium | | | | |
| Silica colloidal | Glidant | 2.00 | 0.72 | 7.201 |
| anhydrous | | | | |
| Magnesium | Lubricant | 6.00 | 2.15 | 21.604 |
| stearate | | | | |
| TOTAL | | 250.00 | (89.45) | (900.196) |
| Film-coating | | | | |
| Opadry amb II | Coating | 2.5 | 0.89 | |
| 88A190022 | agent | | | |
| clear | | | | |
| ACRYL-EZE | Coating | 25.0 | 8.94 | |
| 93A19346 clear | agent | | | |
| PEG8000 | Plasticizer | 2.0 | 0.72 | |
| TOTAL | / | 279.5 | 100 | / |

TABLE 10
Pitolisant HCl formulation for 40 mg gastro-resistant
coated tablets (LF22138-1) - direct compression

| | | Unit | Centesimal | Manufacturing |
|---|---|---|---|---|
| | | formula | formula | formula |
| Raw material | Function | (mg) | (%) | (g) |
| Internal phase | | | | |
| Pitolisant HCl | API | 40.00 | 14.31 | 144.0 |
| (micronized) | | | | |
| Magnesium | Alkaline | 30.00 | 10.73 | 108.1 |
| carbonate | buffer | | | |
| Cellulose | Filler | 53.58 | 19.17 | 192.9 |
| microcrystalline | | | | |
| Tribasic | Filler | 107.17 | 38.34 | 385.9 |
| calcium | | | | |
| phosphate | | | | |
| Croscarmellose | Disintegrant | 11.25 | 4.03 | 40.491 |
| sodium | | | | |
| Silica colloidal | Glidant | 2.00 | 0.72 | 7.201 |
| anhydrous | | | | |
| Magnesium | Lubricant | 6.00 | 2.15 | 21.604 |
| stearate | | | | |
| TOTAL | | 250.00 | (89.45) | (900.196) |
| Film-coating | | | | |
| Opadry amb II | Coating | 2.5 | 0.89 | |
| 88A190022 | agent | | | |
| clear | | | | |
| ACRYL-EZE | Coating | 25.0 | 8.94 | |
| 93A19346 clear | agent | | | |
| PEG8000 | Plasticizer | 2.0 | 0.72 | |
| TOTAL | / | 279.5 | 100 | / |

The batches tested for the stability study are described in Table 11.

TABLE 11
Stability Study Batches

| | Dosage | | | | Batch size (kg |
|---|---|---|---|---|---|
| Bulk product | strength (as | | | Date of | equivalent to |
| batch number | pitolisant HCl) | API manufacturer | API Batch No. | manufacture | tablets) |
| LF22133 | 40 mg | Corden Pharma | MA-22-013 | Aug. 16^{th}, | 841 g |
| | | (Chenôve, France) | | 2022 | |
| LF22134 | 40 mg | Corden Pharma | MA-22-013 | Aug. 17^{th}, | 658 g |
| | | (Chenôve, France) | | 2022 | |
| LF22135 | 40 mg | Corden Pharma | MA-22-013 | Aug. 17^{th}, | 901 g |
| | | (Chenôve, France) | | 2022 | |
| LF22136 | 40 mg | Corden Pharma | MA-22-013 | Aug. 18^{th}, | 700 g |
| | | (Chenôve, France) | | 2022 | |
| LF22137 | 40 mg | Corden Pharma | MA-22-013 | Aug. 19^{th}, | 900 g |
| | | (Chenôve, France) | | 2022 | |
| LF22138 | 40 mg | Corden Pharma | MA-22-013 | Aug. 19^{th}, | 593 g |
| | | (Chenôve, France) | | 2022 | |

The storage of samples was performed according to ICH conditions: long term conditions 25° C./60% RH; intermediate conditions 30° C./65% RH; accelerated conditions 40° C./75% RH; drastic conditions 50° C./75% RH.

Results: Pitolisant Hydrochloride 40 mg, Gastro Resistant Film-Coated Tablets, Batch LF22133-1-A1 after 6 months of Storage at 25° C./60% RH and at 40° C. 75% RH

### Appearance:

All conditions 25° C./60% RH, 30° C./65% RH, 40° C./75% RH and 50° C./75% RH (except at 50° C./75% RH for the T3 months time point) were compared from 15 days to 6 months of storage. No change of appearance was observed after 6 months at 25° C./60% RH. The appearance complied with the specification. Nevertheless, a change of color from white to pink was observed from 15 days to 6 months of storage at 30° C./65% RH and 40° C./75% RH, a more pronounced pink coloration was observed at 50° C./75% RH.

The appearance did not comply with the specification from 15 days to 6 months at 40° C./75% RH and after 3 months at 30° C./65% RH.

From T2 months, some differences were observed between tablets stored in the same condition.

### Mean Mass:

A decrease of the mean mass was observed after 1 month at 25° C./60% RH and at 40° C./75% RH (respectively from 274.5 mg to 272.1 mg and 268.7 mg). From T2 months, the mean mass was not performed. This decrease was correlated with the decrease of the water content. Mean mass complied with the specification.

### Water Content by KF:

A decrease of the water content was observed after 1 month at 25° C./60% RH and at 40° C./75% RH (respectively from 3.2% to 2.5% and 2.2%). This decrease was correlated with the decrease of the mean mass. From T2 months, the water content was not performed.

### Hardness:

No trend was observed for the hardness after 1 month at 25° C./60% RH and at 40° C./75% RH. From T2 months, the hardness was not performed

### Dissolution:

The dissolution results complied with the specification for T1 month at 25° C./60% RH and at 40° C./75% RH without changes from initial, in acid and buffer steps. From T2 months, the dissolution testing was not performed. Heterogeneous results were observed after 15 minutes in buffer pH 6.8 medium (after 135 minutes of dissolution).

### Disintegration Time:

Disintegration results were not compliant, on 6 tablets tested 1 tablet is open and 3 tablets are swollen after 2 hours in acid medium (HCl 0.1M). After 1 month at 25° C./60% RH results complied with the specification, nevertheless disintegration results were not compliant after 1 month at 40° C./75% RH, 3 tablets were swollen. From T2 months, the disintegration time was not performed. Based on the results above, the gastric resistant coating was not optimized for the formulation, resulting in heterogeneity from one unit to another.

### Assay/Purity:

Regarding the assay, no trend was observed after 6 months at 25° C./60% RH, nevertheless a decrease was observed after 6 months at 40° C./75% RH (from 99% to E1=89% and E2=94%). The assay results complied with the specification at 25° C./60% RH, but did not comply at 40° C./75% RH.

Regarding the purity, the main impurities observed after 6 months were: impurity BF4: from <LQ at initial to 0.3% at 25° C./60% RH and 2.5% at 40° C./75% RH; impurity N-Oxide: from <LQ at initial to 0.2% at 25° C./60% RH and 0.5% at 40° C./75% RH; and other impurities: from <LQ at initial to 0.2% at 40° C./75% RH

After 6 months at 25° C./60% RH BF4 impurity was not compliant (this observation will be checked at further stability results). Nevertheless, others impurities as well as total impurities complied with specification. After 6 months at 40° C./75% RH BF4 impurity and total impurities results were not compliant.

### Conclusion

Based on the appearance and the assay/purity results above, the pitolisant hydrochloride 40 mg, gastro resistant film-coated tablets, batch LF22133-1-A1 wet granulation formulation containing not micronized API and magnesium carbonate in HDPE bottles (15 tablets per bottle) was considered not stable at 40° C./75% RH. At 25° C./60% RH results for appearance and assay/purity were compliant except for BF4 impurity.

These results suggest potential interaction of pitolisant with the combination of excipients when exposed to accelerated heat and moisture conditions. Refrigerated (2-8° C.) conditions were not included in this study. This trend concerning purity results was confirmed by similar results obtained with batch LF22134-1-A1 whose formula contains the same excipients, only the API differs (micronized or not).

Results: Pitolisant Hydrochloride 40 mg, Gastro Resistant Film-Coated Tablets, Batch LF22134-1-A1 after 3 Months of Storage at 25° C./60% RH and at 40° C. 75% RH

### Appearance:

All conditions 25° C./60% RH, 30° C./65% RH, 40° C./75% RH and 50° C./75% (except at 50° C./75% RH for the T3 months time point) were compared from 15 days to 3 months of storage. No change of appearance was observed after 3 months at 25° C./60% RH. The appearance complied with the specification. Nevertheless, a change of color from white to pink was observed from 15 days to 3 months of storage at 30° C./65% RH and 40° C./75% RH, a more pronounced pink coloration was observed at 50° C./75% RH.

The appearance did not comply with the specification from 15 days to 3 months at 40° C./75% RH and after 3 months at 30° C./65% RH. From T2 months, some differences were observed between tablets stored in a same condition.

### Mean Mass:

A decrease of the mean mass was observed after 1 month at 25° C./60% RH and at 40° C./75% RH (respectively from 274.2 mg to 271.0 mg and 269.4 mg). From T2 months, the mean mass was not performed. This decrease was correlated with the decrease of the water content. Mean mass complied with the specification.

### Water Content by KF:

A decrease of the water content was observed after 1 month at 25° C./60% RH and at 40° C./75% RH (respectively from 3.0% to 2.4% and 2.1%). This decrease was correlated with the decrease of the mean mass. From T2 months, the water content was not performed.

### Hardness:

No trend was observed for the hardness after 1 month at 25° C./60% RH and at 40° C./75% RH. From T2 months, the hardness was not performed

### Dissolution:

No significant trend was observed between initial results and results after 1 month of stability at 25° C./60% RH and at 40° C./75% RH. The dissolution results complied with the specification.

### Disintegration Time:

Disintegration results were compliant, however, on 6 tablets tested one tablet was swollen after 2 hours in acid medium (HCl 0.1M). After 1 month at 25° C./60% RH and 40° C./75% RH results complied with the specification. From T2 months, the disintegration time was not performed.

### Assay/Purity:

Regarding the assay, a slight decrease was observed after 3 months at 25° C./60% RH and at 40° C./75% RH (respectively from 98% to 96% and 92%). The assay results were not compliant with the specification at 25° C./60% RH after 2 months and at 40° C./75% RH after 3 months.

Regarding the purity, the main impurities observed after 3 months are: impurity BF4: from <LQ at initial to 0.1% at 25° C./60% RH and 0.7% at 40° C./75% RH; impurity N-Oxide: from <LQ at initial to 0.1% at 25° C./60% RH and 0.2% at 40° C./75% RH; other impurities: from <LQ at initial to 0.1% at 40° C./75% RH.

Individual impurity as well as total impurities complied with specification at 25° C./60% RH. BF4 impurity and total impurities results were not compliant at 40° C./75% RH.

### Conclusion

Based on the appearance, assay/purity results above, the pitolisant hydrochloride 40 mg, gastro resistant film-coated tablets, batch LF22134-1-A1 wet granulation formulation containing micronized API and magnesium carbonate in HDPE bottles (15 tablets per bottle) was considered not stable at 40° C./75% RH, but was within specification at room temperature (25° C./60% RH) except for assay results up to 3 months.

These results suggest potential interaction of pitolisant with the combination of excipients when exposed to accelerated heat and moisture conditions. Refrigerated (2-8° C.) conditions were not included in this study. This trend was confirmed by similar results obtained with batch LF22133-1-A1 whose formula contains the same excipients, only the API (micronized or not) differs. The stability study for this batch (containing micronized API) was stopped after the T3 month time point.

Results: Pitolisant Hydrochloride 40 mg, Gastro Resistant Film-Coated Tablets, Batch LF22135-1-A1 after 6 months of Storage at 25° C./60%/RH and at 40° C. 75% RH

### Appearance:

All conditions 25° C./60% RH, 30° C./65% RH, 40° C./75% RH and 50° C./75% RH (except at 50° C./75% RH for the T3 months time point) were compared from 15 days to 6 months of storage. No change in appearance was observed after 6 months at 25° C./60% RH and 30° C./65% RH. The appearance complied with the specification. Nevertheless, a slight pink coloration was observed from 15 days to 6 months of storage at 40° C./75% RH. The appearance did not comply with the specification from 15 days to 6 months at 40° C./75% RH, a more pronounced pink coloration was observed at 50° C./75% RH. After 3 months, some differences were observed between tablets stored in a same condition.

### Mean Mass:

A decrease of the mean mass was observed after 1 month at 25° C./60% RH and at 40° C./75% RH (respectively from 275.3 mg to 271.5 mg and 269.9 mg). From T2 months, the mean mass was not performed. This decrease was correlated with the decrease of the water content. Mean mass complied with the specification.

### Water Content by KF:

A decrease of the water content was observed after 1 month at 25° C./60% RH and at 40° C./75% RH (respectively from 2.8% to 2.0% and 1.8%). This decrease was correlated with the decrease of the mean mass. From T2 months, the water content was not performed.

### Hardness:

No trend was observed for the hardness after 1 month at 25° C./60% RH and at 40° C./75% RH. From T2 months, the hardness was not performed

### Dissolution:

Dissolution profiles at initial and after 1 month at 25° C./60% RH and at 40° C./75% RH were similar. Dissolution results were not compliant with the specification for T1 month at 25° C./60% RH and at 40° C./75% RH without changes from initial, in acid and buffer steps. From T2 months, the dissolution testing was not performed. Heterogeneous results were observed after 15 minutes in buffer pH 6.8 medium (after 135 minutes of dissolution).

### Disintegration Time:

At initial and after 1 month at 25° C./60% RH and 40° C./65% RH, disintegration results were not compliant after 2 hours in acid medium (HCl 0.1M). From T2 months, the disintegration time was not performed. Based on the results above, the coating levels were not optimal for the formulation, resulting in heterogeneity from one unit to another.

### Assay/Purity:

Regarding the assay, a slight decrease was observed after 6 months at 25° C./60% RH and at 40° C./75% RH (respectively from 99% to 97% and 95%). The assay results complied with the specification at 25° C./60% RH but did not comply at 40° C./75% RH. Regarding the purity, the main impurities observed after 6 months are: impurity BF4: from <LQ at initial to 0.1% at 25° C./60% HR, 1.2% at 40° C./75% RH; impurity N-Oxide: from <LQ at initial to 0.1% at 25° C./60% HR, 0.5% at 40° C./75% RH; and impurity at RRT 0.43: from <LQ at initial to 0.5% at 40° C./75% RH. Individual impurity, as well as total impurities, complied with specification at 25° C./60% RH. Nevertheless, at 40° C./75% RH all the purity results were not compliant.

### Conclusion

For the samples that were stored at accelerated conditions, results above show that the pitolisant hydrochloride 40 mg, gastro resistant film-coated tablets, batch LF22135-1-A1 direct compression formulation containing not micronized API and Meglumine in HDPE bottles (15 tablets per bottle) were considered not stable.

The appearance and assay/purity results seem to be explained by potential interactions of pitolisant with the combination of excipients when exposed to high heat and moisture. Nevertheless, for the samples stored at 25° C./60% RH, dissolution and disintegration results show that the formulation of pitolisant hydrochloride 40 mg, gastro resistant film-coated tablets, batch LF22135-1-A1 direct compression formulation containing not micronized API and Meglumine in HDPE bottles (15 tablets per bottle) was not optimal.

Results: Pitolisant Hydrochloride 40 mg, Gastro Resistant Film-Coated Tablets, Batch LF22136-1-A1 after 3 Months of Storage at 25° C./60%/RH and at 40° C. 75% RH

### Appearance:

All conditions 25° C./60% RH, 30° C./65% RH, 40° C./75% RH and 50° C./75% RH (except at 50° C./75% RH for the T3 months time point) were compared from 15 days to 3 months of storage. No change in appearance was observed after 3 months at 25° C./60% RH. The appearance complies with the specification. Nevertheless, a change of color from white to pink was observed from 15 days to 3 months of storage at 40° C./75% RH and after 3 months at 30° C./65% RH, a more pronounced pink coloration was observed at 50° C./75% RH.

The appearance did not comply with the specification from 15 days to 3 months at 40° C./75% RH and after 3 months at 30° C./65% RH. After T3 months, some differences were observed between tablets stored in a same condition.

### Mean Mass:

A decrease of the mean mass was observed after 1 month at 25° C./60% RH and at 40° C./75% RH (respectively from 275.0 mg to 272.4 mg and 272.0 mg). From T2 months, the mean mass was not performed. This decrease was correlated with the decrease of the water content. Mean mass complied with the specification.

### Water Content by KF:

A decrease of the water content was observed after 1 month at 25° C./60% RH and at 40° C./75% RH (respectively from 2.8% to 2.1% and 1.7%). This decrease was correlated with the decrease of the mean mass. From T2 months, the water content was not performed.

### Hardness:

A slight increase was observed for the hardness after 1 month at 25° C./60% RH and at 40° C./75% RH (respectively from 104N to 122N and 112N). From T2 months, the hardness was not performed.

### Dissolution:

In acid media, the dissolution results complied with the specification after 1 month at 25° C./60% RH and at 40° C./75% RH without changes from initial. In buffer step, stage 1, the dissolution results were not compliant with the specification after 1 month at 25° C./60% RH (2 units <80% after 30 minutes in buffer medium) and at 40° C./75% RH (1 unit <80% after 30 minutes in buffer medium) without changes from initial results (1 unit <80% after 30 minutes in acid medium). From T2 months, the dissolution testing was not performed.

### Disintegration Time:

No change was observed for the disintegration time after 1 month at 25° C./60% RH and at 40° C./75% RH. From T2 months, the disintegration time was not performed. The disintegration time results complied with the specification.

### Assay/Purity:

Regarding the assay, the initial value was near the lower acceptance criteria, after 3 months at 25° C./60% RH and at 40° C./75% RH no trend was observed. The assay results complied with the initial results but did not comply with the specification after 1 and 2 months at 40° C./75% RH.

Regarding the purity, the main impurities observed after 3 months are: impurity BF4: from <LQ at initial to 0.1% at 25° C./60% RH and 0.3% at 40° C./75% RH; impurity N-Oxide: from <LQ at initial to 0.1% at 25° C./60% RH and 0.2% at 40° C./75% RH and impurity at RRT 0.38: from <LQ at initial to 0.2% at 40° C./75% RH.

Individual impurity as well as total impurities complied with specification at 25° C./60% RH. BF4 impurity results were not compliant at 40° C./75% RH.

### Conclusion

Based on the appearance; assay and dissolution results above, the pitolisant hydrochloride 40 mg, gastro resistant film-coated tablets, batch LF22136-1-A1 direct compression formulation containing micronized API and Meglumine in HDPE bottles (15 tablets per bottle) was considered not stable at 40° C./75% RH, but were within specification at room temperature (25° C./60% RH), except for dissolution testing which was not compliant (level 1) at initial and after 1 month at 25° C./60% RH.

These results seem to be explained by heterogeneity of gastro-resistant coating (non-optimized coating) from one unit to another and potential interactions under exposure of accelerated conditions. Refrigerated (2-8° C.) conditions were not included in this study.

The stability study for this batch (containing micronized API) was stopped after the T3 month time point

Results: Pitolisant Hydrochloride 40 mg, Gastro Resistant Film-Coated Tablets, Batch LF22137-1-A1 after 6 Months of Storage at 25° C./60%/RH and at 40° C. 75% RH

### Appearance:

All conditions 25° C./60% RH, 30° C./65% RH, 40° C./75% RH and 50° C./75% RH (except at 50° C./75% RH for the T3 months time point) were compared from 15 days to 6 months of storage. No change in appearance was observed after 1 month at 25° C./60% RH. Nevertheless, a change of color from white to pink was observed from 2 months and from 15 days to 6 months of storage at 30° C./65% RH, 40° C./75% RH and 50° C./75% RH, a more pronounced pink coloration was observed at 50° C./75% RH.

The appearance did not comply with the specification from 3 months at 25° C./60% RH and from 15 days to 6 months at 40° C./75% RH. From T2 months, some differences were observed between tablets stored in a same condition.

### Mean Mass:

A decrease of the mean mass was observed after 1 month at 25° C./60% RH and at 40° C./75% RH (respectively from 275.4 mg to 272.2 mg and 271.6 mg). From T2 months, the mean mass was not performed. This decrease was correlated with the decrease of the water content. Mean mass complied with the specification.

### Water Content by KF:

A decrease of the water content was observed after 1 month at 25° C./60% RH and at 40° C./75% RH (respectively from 3.4% to 2.4% and 2.2%). This decrease was correlated with the decrease of the mean mass. From T2 months, the water content was not performed.

### Hardness:

No trend was observed for the hardness after 1 month at 25° C./60% RH and at 40° C./75% RH. From T2 months, the hardness was not performed

### Dissolution:

No trend was observed in dissolution profiles after 1 month at 25° C./60% RH and at 40° C./75% RH. The dissolution results complied with the specification without changes from initial, in acid and buffer steps. From T2 months, the dissolution testing was not performed.

### Disintegration Time:

At initial, disintegration results were not compliant, on 6 tablets tested 1 tablet is open and 1 tablet is swollen after 2 hours in acid medium (HCl 0.1M). After 1 month at 25° C./60% RH and at 40° C./75% RH results complied with the specification, however at 40° C./75% RH on 6 tablets tested 4 tablets were swollen. From T2 months, the disintegration time was not performed.

Based on the results above, the coating will be further developed and optimized for the formulation, to overcome heterogeneity from one unit to another.

### Assay/Purity:

Regarding the assay, a slight decrease was observed after 6 months at 25° C./65% HR and 40° C./75% RH. The assay results complied with the specification at 25° C./60% RH and at 40° C./75% RH with an out of specification result after 1 month at 40° C./75% RH.

Regarding the purity, the main impurities observed after 6 months were: impurity BF4: from <LQ at initial to 0.6% at 25° C./60% RH and 1.3% at 40° C./75% RH; impurity N-Oxide: from <LQ at initial to 0.1% at 25° C./60% RH and 0.2% at 40° C./75% RH; and other impurities: from <LQ at initial to 0.1% at 40° C./75% RH.

After 6 months at 25° C./60% RH BF4 impurity was not compliant (this observation will be checked at further stability results). Nevertheless, others impurities as well as total impurities complied with specification.

After 6 months at 40° C./75% RH BF4 impurity and total impurities results were not compliant.

### Conclusion

Based on the appearance, disintegration, assay, and impurity results above, the pitolisant hydrochloride 40 mg, gastro resistant film-coated tablets, batch LF22137-1-A1 direct compression formulation containing not micronized API and Magnesium Carbonate in HDPE bottles (15 tablets per bottle) was considered not stable at 40° C./75% RH, but were within specification at room temperature (25° C./60% RH) except for appearance which were not compliant after 2 months at 25° C./60% RH and the BF4 impurity, not compliant after 6 months at 25° C./60% RH.

The disintegration results seem to be explained by heterogeneity of gastro resistant coating (non-optimized coating) from one unit to another. The appearance, assay and impurity results seem to be explained by potential interactions under exposure of accelerated conditions. Refrigerated (2-8° C.) conditions were not included in this study.

Results: Pitolisant Hydrochloride 40 mg, Gastro Resistant Film-Coated Tablets, Batch LF22138-1-A1 after 3 Months of Storage at 25° C./60%/RH and at 40° C. 75% RH

### Appearance:

All conditions 25° C./60% RH, 30° C./65% RH, 40° C./75% RH and 50° C./75% RH (except at 50° C./75% RH for the T3 months time point) were compared from 15 days to 3 months of storage. An important change of color from white to pink was observed from 15 days to 3 months of storage at all the conditions, a more pronounced pink coloration was observed at 50° C./75% RH.

The appearance did not comply with the specification from 15 days to 3 months. From T2 months, some differences were observed between tablets stored in a same condition.

### Mean Mass:

No change in Mean Mass was observed after 1 months at 25° C./60% RH. A decrease of the mean mass was observed after 1 month at 40° C./75% RH (from 273.4 mg to 271.2 mg). From T2 months, the mean mass was not performed. This decrease was correlated with the decrease of the water content. Mean mass complied with the specification.

### Water Content by KF:

A decrease of the water content was observed after 1 month at 25° C./60% RH and at 40° C./75% RH (respectively from 2.9% to 2.2% and 2.1%). This decrease was correlated with the decrease of the mean mass. From T2 months, the water content was not performed.

### Hardness:

No trend was observed for the hardness after 1 month at 25° C./60% RH and at 40° C./75% RH. From T2 months, the hardness was not performed

### Dissolution:

No trend was observed in dissolution profiles after 1 month at 25° C./60% RH and at 40° C./75% RH. The dissolution results complied with the specification without changes from initial, in acid and buffer steps. From T2 months, the dissolution testing was not performed.

### Disintegration Time:

At initial, disintegration results complied with specifications. After 15 days and 1 month at 25° C./60% RH and 40° C./75% RH, some disintegration results were not compliant after 2 hours in acid medium (HCl 0.1M) due to opening of one or more tablets during testing.

Based on the results above, the coating seems to be not suitable for the formulation, resulting in heterogeneity from one unit to another.

### Assay/Purity:

Regarding the assay, no trend was observed after 3 months at 25° C./60% RH, however, a slight decrease was observed after 3 months at 40° C./75% RH (from 96% to 94%). The assay results did not comply with the specification at 25° C./60% RH and at 40° C./75% RH.

Regarding the purity, the main impurities observed after 3 months are: impurity BF4: from <LQ at initial to 0.2% at 25° C./60% RH and 0.4% at 40° C./75% RH; impurity N-Oxide: from <LQ at initial to 0.2% at 40° C./75% RH; and other impurities: from <LQ at initial to 0.1% at 40° C./75% RH.

Individual impurity, as well as total impurities, complied with specification at 25° C./60% RH. BF4 impurity results were not compliant at 40° C./75% RH.

### Conclusion

Based on the appearance, assay and disintegration results above, the pitolisant hydrochloride 40 mg, gastro resistant film-coated tablets, batch LF22136-1-A1 direct compression formulation containing micronized API and Magnesium carbonate in HDPE bottles (15 tablets per bottle) was considered not stable at 40° C./75% RH, but were within specification at room temperature (25° C./60% RH) except for appearance and disintegration results which were not compliant after 1 and 2 months at 25° C./60% RH.

These results seem to be explained by heterogeneity of gastro resistant coating (non-optimized coating) from one unit to another and potential interactions under exposure of accelerated conditions. Refrigerated (2-8° C.) conditions were not included in this study.

The stability study for this batch (containing micronized API) was stopped after the T3 month time point

### Example 3. Three Month Supportive Stability Study

Four supportive batches were manufactured by Synerlab Developpement using optimized GR film-coating (Acryleze 15%, AMB 3%, PEG 8%) with three different alkaline agents: magnesium carbonate (high dose—30 g and low dose 7.5 mg), potassium bicarbonate (30 mg), and sodium carbonate (30 mg).

TABLE 12
Pitolisant HCl 40 mg gastro-resistant film-coated tablets formulations

| | Raw material | | | |
|---|---|---|---|---|
| | Unit formula | Unit formula | Unit formula | Unit formula |
| | (mg/unit) | (mg/unit) | (mg/unit) | (mg/unit) |
| | Formulation number | | | |
| | Formulation | Formulation | Formulation | Formulation |
| | no1 | no2 | no3 | no4 |
| | Batch number | | | |
| | LF23061-2A1 | LF23066-2A1 | LF23067-2A1 | LF23070-2A1 |
| Pitolisant HCl | 40.00^{a} | 40.00^{a} | 40.00^{a} | 40.00^{a} |
| Magnesium carbonate | 30.00 | 7.50 | / | / |
| Potassium bicarbonate | / | / | 30.00 | / |
| Sodium carbonate | / | / | / | 30.00 |
| Lactose | 107.17 | 122.17 | 107.17 | 107.17 |
| Microcrystalline cellulose | 53.58 | 61.08 | 53.58 | 53.58 |
| Croscarmellose sodium | 11.25 | 11.25 | 11.25 | 11.25 |
| Anhydrous colloidal silica | 2.00 | 2.00 | 2.00 | 2.00 |
| Magnesium stearate | 6.00 | 6.00 | 6.00 | 6.00 |
| TOTAL | 250.0 | 250.0 | 250.0 | 250.0 |
| Coating | | | | |
| Opadry amb II¹ (clear, | 7.5 | 7.5 | 7.5 | 7.5 |
| 88A190022) | | | | |
| Acryl eze² 93A19346 clear | 38.625 | 38.625 | 38.625 | 38.625 |
| PEG 8000³ | 3.09 | 3.09 | 3.09 | 3.09 |
| TOTAL | 299.215 | 299.215 | 299.215 | 299.215 |

^{a}Salt/Base ratio = 1.1234
¹15% suspension, weight gain = 3%
²20% suspension, weight gain = 15%
³PEG at 8% of acryl eze

The objectives of this stability study were to define a shelf-life period to support future clinical studies after coating optimization, and to generate stability data for the IMPD of the gastro-resistant coated tablets in HDPE bottles (15 tablets per bottle).

This report summarizes the data obtained after 3 months of storage; under ICH conditions at 5° C., 25° C./60% RH, 30° C./65% RH and 40° C./75% RH and under drastic conditions 50° C./75% RH.

TABLE 13A
NP1 supportive batch (LF23061) for IMPD (T2 months at 5° C. and 25° C./60% RH); T3 months

| | | | LF23061 (magnesium | | | |
|---|---|---|---|---|---|---|
| | | Acceptance criteria | carbonate 30 mg) | | | |
| Disintegration | Results in HCl 0.1M for 2 | Complies | Complies | | | |
| (n = 6) | hours without disks: | No tablet shows signs of either | Complies | | | |
| | After 1 hour | disintegration (apart from | 5° | C. | 25° | C. |
| | After 2 hours | fragments of coating) or cracks | | | | |
| | | that would allow the escape of | | | | |
| | | contents | | | | |
| | In Buffer Stage (pH 6.8) for | Comply to Eur. Ph. 2.9.1 | 25 | min | 27 | min |

| | | | | |
|---|---|---|---|---|
| | 1 hour (with disks) [Max] | | | |
| Dissolution | Results (%) dissolved | Stage 1: No individuals value | 0 (6) [0-0] | 0 (6) [0-0] |
| (% pitolisant | In acid (HCl 0.1M) after 120 | exceeds 10% dissolved at 120 | | |
| dissolved) | minutes | min | | |
| | Average (n) [min-max] | | | |
| | In buffer stage (pH 6.8) | Stage 1: No unit ≤ Q + 5% | 30 min: 17% | 30 min: 31% |
| | Average (n = 6) [min-max] | (80%) after 45 mins | 5 to 40 | 15 to 63 |
| | | Stage 2 and 3: | 45 min: 98% | 45 min: 98% |
| | | see Eur. Ph. 2.9.3/USP <711> | 95 to 101 | 95 to 100 |
| Water content | Mean (%) | ≤5% | 2.4% | 2.2% |
| Assay | % | 95.0-105.0 | 98 (97.6) | 99 (98.5) |
| Impurities | % | Total of impurity by LC ≤1.0% | <0.1 | <0.1 |

TABLE 13B
NP1 supportive batch (LF23066) for IMPD (T2 months at 5° C. and 25° C./60% RH); T3 months

| | | | LF23066 (magnesium | | | |
|---|---|---|---|---|---|---|
| | | Acceptance criteria | carbonate 7.5 mg) | | | |
| Disintegration | Results in HCl 0.1M for 2 | Complies | Complies | | | |
| (n = 6) | hours without disks: | No tablet shows signs of either | Complies | | | |
| | After 1 hour | disintegration (apart from | 5° | C. | 25° | C. |
| | After 2 hours | fragments of coating) or cracks | | | | |
| | | that would allow the escape of | | | | |
| | | contents | | | | |
| | In Buffer Stage (pH 6.8) for | Comply to Eur. Ph. 2.9.1 | 24 | min | 27 | min |

| | | | | |
|---|---|---|---|---|
| | 1 hour (with disks) [Max] | | | |
| Dissolution | Results (%) dissolved | Stage 1: No individuals value | 0 (6) [0-0] | 0 (6) [0-0] |
| (% pitolisant | In acid (HCl 0.1M) after 120 | exceeds 10% dissolved at 120 | | |
| dissolved) | minutes | min | | |
| | Average (n) [min-max] | | | |
| | In buffer stage (pH 6.8) | Stage 1: No unit ≤ Q + 5% | 30 min: 46% | 30 min: 42% |
| | Average (n = 6) [min-max] | (80%) after 45 mins | 13 to 77 | 19 to 70 |
| | | Stage 2 and 3: | 45 min: 99% | 45 min: 99% |
| | | see Eur. Ph. 2.9.3/USP <711> | 98 to 100 | 98 to 99 |
| Water content | Mean (%) | ≤5% | 2.2% | 2.3% |
| Assay | % | 95.0-105.0 | 99 (98.9) | 98 (97.9) |
| Impurities | % | Total of impurity by LC ≤1.0% | <0.1 | <0.1 |

TABLE 13C
NP1 supportive batch (LF23067) for IMPD (T2 months at 5° C. and 25° C./60% RH); T3 months

| | | | LF23067 (potassium | | | |
|---|---|---|---|---|---|---|
| | | Acceptance criteria | bicarbonate 30 mg) | | | |
| Disintegration | Results in HCl 0.1M for 2 | Complies | Complies | | | |
| (n = 6) | hours without disks: | No tablet shows signs of either | Complies | | | |
| | After 1 hour | disintegration (apart from | 5° | C. | 25° | C. |
| | After 2 hours | fragments of coating) or cracks | | | | |
| | | that would allow the escape of | | | | |
| | | contents | | | | |
| | In Buffer Stage (pH 6.8) for | Comply to Eur. Ph. 2.9.1 | 28 | min | 30 | min |

| | | | | |
|---|---|---|---|---|
| | 1 hour (with disks) [Max] | | | |
| Dissolution | Results (%) dissolved | Stage 1: No individuals value | 0 (6) [0-0] | 0 (6) [0-0] |
| (% pitolisant | In acid (HCl 0.1M) after 120 | exceeds 10% dissolved at 120 | | |
| dissolved) | minutes | min | | |
| | Average (n) [min-max] | | | |
| | In buffer stage (pH 6.8) | Stage 1: No unit ≤ Q + 5% | 30 min: 55% | 30 min: 45% |
| | Average (n = 6) [min-max] | (80%) after 45 mins | 39 to 73 | 30 to 59 |
| | | Stage 2 and 3: | 45 min: 99% | 45 min: 98% |
| | | see Eur. Ph. 2.9.3/USP <711> | 98 to 102 | 96 to 99 |
| Water content | Mean (%) | ≤5% | 3.6% | 3.6% |
| Assay | % | 95.0-105.0 | 97 (97.1) | 98 (97.7) |
| Impurities | % | Total of impurity by LC ≤1.0% | <0.1 | <0.1 |

TABLE 13D
NP1 supportive batch (LF23070) for IMPD (T2 months at 5° C. and 25° C./60% RH); T3 months

| | | | LF23070 (sodium | | | |
|---|---|---|---|---|---|---|
| | | Acceptance criteria | carbonate 30 mg) | | | |
| Disintegration | Results in HCl 0.1M for 2 | Complies | Complies | | | |
| (n = 6) | hours without disks: | No tablet shows signs of either | Complies | | | |
| | After 1 hour | disintegration (apart from | 5° | C. | 25° | C. |
| | After 2 hours | fragments of coating) or cracks | | | | |
| | | that would allow the escape of | | | | |
| | | contents | | | | |
| | In Buffer Stage (pH 6.8) for | Comply to Eur. Ph. 2.9.1 | 52 | min | 31 | min |

| | | | | |
|---|---|---|---|---|
| | 1 hour (with disks) [Max] | | (52-43-27- | (29-28-29- |
| | | | 27-25-25) | 28-27-31) |
| Dissolution | Results (%) dissolved | Stage 1: No individuals value | 0 (6) [0-0] | 0 (6) [0-0] |
| (% pitolisant | In acid (HCl 0.1M) after 120 | exceeds 10% dissolved at 120 | | |
| dissolved) | minutes | min | | |
| | Average (n) [min-max] | | | |
| | In buffer stage (pH 6.8) | Stage 1: No unit ≤ Q + 5% | 30 min: 16% | 30 min: 13% |
| | Average (n = 6) [min-max] | (80%) after 45 mins | 9 to 23 | 10 to 18 |
| | | Stage 2 and 3: | 45 min: 95% | 45 min: 92% |
| | | see Eur. Ph. 2.9.3/USP <711> | 93 to 97 | 80 to 98 |
| Water content | Mean (%) | ≤5% | 3.4% | 2.3% |
| Assay | % | 95.0-105.0 | 98 (97.8) | 98 (97.9) |
| Impurities | % | Total of impurity by LC ≤1.0% | <0.1 | <0.1 |

Following the initial analysis of supportive batches (LF23061-2, LF23066-2, LF23067-2 and LF23070-2) manufactured with 3 different alkaline agents: magnesium carbonate (at 30 mg and 7.5 mg), potassium bicarbonate (at 30 mg) and sodium carbonate (at 30 mg) by direct compression and film-coated with gastro resistant film, this report details results obtained after T3M of stability study.

Regarding the batch LF23061-2 (magnesium carbonate 30 mg), appearance, mean mass, water content (by KF volumetry), Assay/Purity and Disintegration (reported for information) were compliant.

Concerning the dissolution at T3M, results complied with the specification at 60 minutes. Whatever the conditions, at initial and during stability, the sampling at 30 minutes of dissolution in buffer media showed heterogeneous RSD.

For the batch LF23066-2 (magnesium carbonate 7.5 mg), appearance, mean mass, water content (KF by volumetry), Assay/Purity and Disintegration (reported for information) were compliant. Dissolution results complied with the specification at 60 minutes. Whatever the conditions, at initial and during stability, the sampling at 30 minutes of dissolution in buffer media showed heterogeneous RSD.

Concerning the batch LF23067-2 (Potassium bicarbonate 30 mg), mean mass, water content (KF by volumetry), Assay/Purity and Disintegration (reported for information) were compliant. Appearance of the tablet complied except after 3 months at 50° C./75% RH, tablets were slightly pink and some tablets were stuck together with a red sticky material. Dissolution results complied with the specification at 60 minutes. However, a slow-down profile was observed after 30 minutes of dissolution in buffer media since T1M stability time point. However, a slow-down profile was observed after 30 minutes of dissolution in buffer media since T1M stability time point. Nevertheless, there were no changes at T2M and T3M regarding results obtained (dissolution profile is similar between the T1M, T2M and T3M stability time points, no additional slow-down profile).

Finally, regarding the batch LF23070-2 (sodium carbonate 30 mg), mean mass, Water content (KF by volumetry), assay/purity and Disintegration (reported for information) were compliant. Appearance of the tablet complied except after 3 months at 50° C./75% RH, a slight pink coloration appeared. Dissolution was compliant with the specification at 60 minutes. For the T3M stability time point, the slow-down profile observed since the T1M stability time point was confirmed. Dissolution profile was similar between the T1M, T2M and T3M stability time points, no additional slow-down profiles.

### Example 4. Alkaline Agents Screening Study: Caco2 Permeability Assay

An in vitro permeability assay was run using Caco2 (human colon adenocarcinoma cells) cells, which spontaneously differentiate into monolayers in polarized enterocytes. After 21 days, differentiated/confluent cell culture batches were assayed and the permeability coefficient Papp (cm/s) was determined.

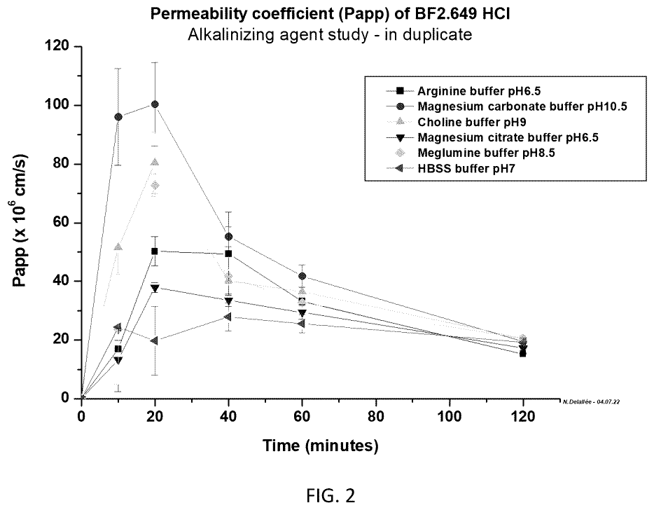

Six different formulations of pitolisant hydrochloride (814 uM local [conc] (60 mg in 250 mL water)) were used to compare the kinetics of five different alkaline agents (magnesium carbonate, meglumine, magnesium citrate, choline, and arginine) against standard HBSS buffer at pH 7 for in vitro permeability assays (see FIG. 2).

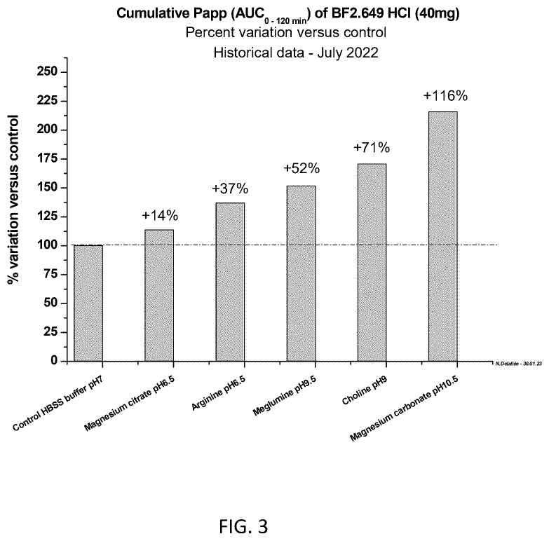

The percent variation of the five different pitolisant hydrochloride formulations containing alkaline agents (magnesium citrate pH 6.5, arginine pH 6.5, meglumine pH 9.5, choline pH 9, and magnesium carbonate pH 10.5) was compared against the control HBSS buffer at pH 7 (see FIG. 3).

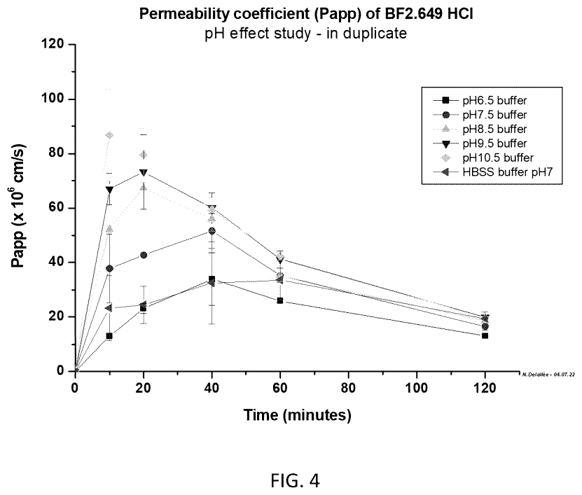

The effect of pH on the permeability coefficient (Papp) was studied using five different buffers (pH 6.5, pH 7.5, pH 8.5, pH 9.5, and pH 10.5) in comparison to the control HBSS buffer at pH 7 (see FIG. 4).

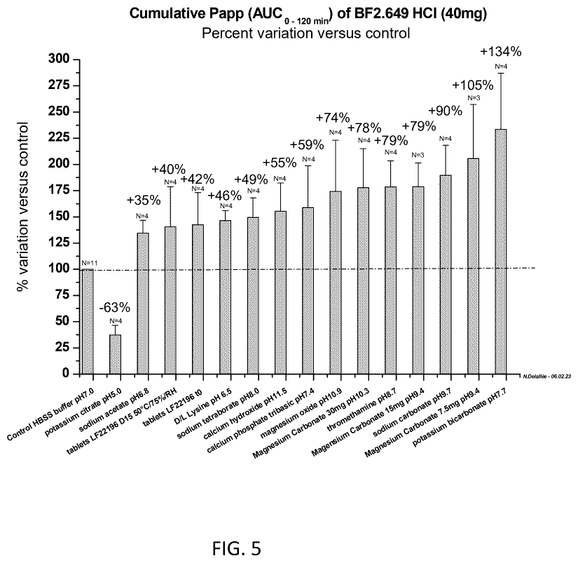

Ten other alkaline agents were evaluated, calcium hydroxide, calcium phosphate tribasic, D/L lysine, magnesium oxide, potassium bicarbonate, potassium citrate, sodium acetate, sodium carbonate, sodium tetraborate, tromethamine (Tris) and magnesium carbonate). Pitolisant 40 mg (tablet strength) with 30 mg alkaline agent in 250 mL water (i.e., 540 uM [local pitolisant]) was used to generate pH measurement and Papp (kinetic study) determination (n=3 to 4) (see FIG. 5).

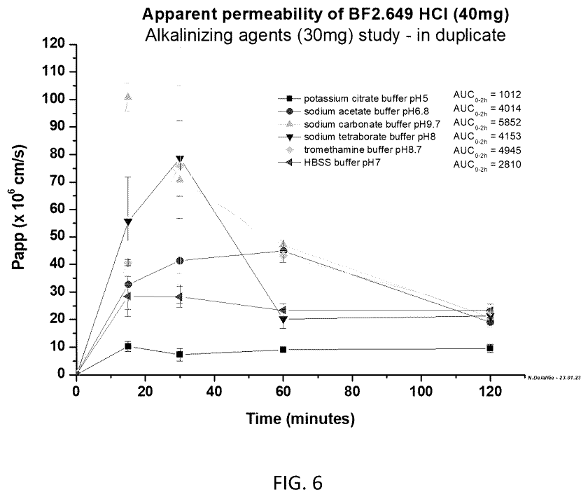

The apparent permeability of pitolisant hydrochloride (40 mg) in combination with an alkaline agent (potassium citrate, sodium acetate, sodium carbonate, sodium tetraborate, or tromethamine) (30 mg) was analyzed and the AUC was calculated (see FIG. 6).

### Example 5. Phase I Clinical Study Comparing Exemplary Dose Forms to WAKIX®

A phase I, open label, single-center, cross-over randomized, relative bioavailability study was conducted, which compared four exemplary dose forms of the present disclosure to WAKIX® as a reference dose form, where single oral administrations were provided to healthy male subjects.

The following exemplary dose forms were prepared according to the present disclosure, and labeled for purposes of this study as “Treatment A,” “Treatment B,” “Treatment C,” and “Treatment D.” Also, for purposes of this study, the reference dose form WAKIX® was labeled “Treatment E.” A description of the pitolisant and alkaline agent content (if applicable) is provided in Table 14 below.

TABLE 14
Description of exemplary dose forms and
reference dose form for clinical study.

| Dose Form Name | Dose Form Description |
|---|---|
| Treatment A | One 40 mg pitolisant hydrochloride tablet containing |
| | 30 mg magnesium carbonate. |
| Treatment B | One 40 mg pitolisant hydrochloride tablet containing |
| | 7.5 mg magnesium carbonate. |
| Treatment C | One 40 mg pitolisant hydrochloride tablet containing |
| | 30 mg potassium bicarbonate |
| Treatment D | One 40 mg pitolisant hydrochloride tablet containing |
| | 30 mg sodium carbonate |
| Treatment E | Two 20 mg pitolisant hydrochloride WAKIX ® tablets |
| (reference) | (no alkaline agent). |

After administering a single oral dose to the subjects, serum was collected and various pharmacokinetic parameters were obtained and analyzed, over a 96 hour period. The serum concentrations obtained are provided in Tables 15 and 16. Results of pharmacokinetic analyses are provided below in Tables 17-19.

TABLE 15
Descriptive statistics of pitolisant serum concentrations
(ng/mL) by treatment - Treatments A and B.

| Treatment | |
|---|---|
| A | B |

| Time | | Mean | SD | | Mean | SD |
|---|---|---|---|---|---|---|
| (h) | N | (ng/mL) | (ng/mL) | N | (ng/mL) | (ng/mL) |
| 0.00 | 15 | 0.00 | 0.00 | 16 | 0.01 | 0.04 |
| 0.25 | 15 | 0.00 | 0.00 | 16 | 0.01 | 0.04 |
| 0.50 | 15 | 0.01 | 0.03 | 16 | 0.01 | 0.04 |
| 0.75 | 14 | 0.00 | 0.00 | 16 | 0.01 | 0.04 |
| 1.00 | 14 | 0.03 | 0.08 | 16 | 0.01 | 0.04 |
| 1.25 | 13 | 0.03 | 0.12 | 16 | 0.05 | 0.16 |
| 1.50 | 13 | 0.14 | 0.51 | 16 | 0.22 | 0.76 |
| 1.75 | 13 | 0.58 | 2.08 | 16 | 1.28 | 4.85 |
| 2.00 | 13 | 1.15 | 4.10 | 16 | 1.84 | 6.83 |
| 2.25 | 14 | 1.15 | 4.11 | 16 | 2.79 | 10.02 |
| 2.50 | 14 | 1.55 | 5.35 | 16 | 3.74 | 11.59 |
| 2.75 | 14 | 1.82 | 5.71 | 16 | 6.81 | 15.60 |
| 3.00 | 14 | 2.73 | 7.37 | 16 | 9.78 | 18.71 |
| 3.50 | 15 | 12.64 | 16.75 | 16 | 22.54 | 27.57 |
| 4.00 | 15 | 21.49 | 19.52 | 16 | 39.45 | 25.89 |
| 6.00 | 15 | 33.75 | 15.82 | 16 | 34.42 | 19.32 |
| 8.00 | 15 | 26.06 | 12.40 | 16 | 25.20 | 14.67 |
| 12.00 | 15 | 16.86 | 8.77 | 16 | 15.35 | 9.68 |
| 24.00 | 15 | 7.88 | 4.83 | 16 | 7.49 | 5.55 |
| 48.00 | 15 | 2.82 | 2.22 | 16 | 2.64 | 2.19 |
| 72.00 | 15 | 1.27 | 1.08 | 16 | 1.16 | 1.07 |
| 96.00 | 14 | 0.67 | 0.61 | 14 | 0.72 | 0.65 |

TABLE 16
Descriptive statistics of pitolisant serum concentrations
(ng/mL) by treatment - Treatments C, D and E.

| | Treatment | | | | | | | | |
|---|---|---|---|---|---|---|---|---|---|
| | C | | | D | | | E | | |
| Time | | Mean | SD | | Mean | SD | | Mean | SD |
| (h) | N | (ng/mL) | (ng/mL) | N | (ng/mL) | (ng/mL) | N | (ng/mL) | (ng/mL) |
| 0.00 | 16 | 0.02 | 0.05 | 16 | 0.03 | 0.07 | 16 | 0.02 | 0.04 |
| 0.25 | 15 | 0.02 | 0.05 | 16 | 0.03 | 0.06 | 16 | 0.21 | 0.22 |
| 0.50 | 16 | 0.05 | 0.07 | 15 | 0.03 | 0.06 | 16 | 0.61 | 0.48 |
| 0.75 | 14 | 0.06 | 0.13 | 14 | 0.03 | 0.06 | 16 | 2.99 | 4.81 |
| 1.00 | 15 | 0.13 | 0.25 | 15 | 0.06 | 0.09 | 16 | 8.08 | 11.47 |
| 1.25 | 15 | 0.39 | 0.77 | 15 | 0.10 | 0.12 | 16 | 14.40 | 16.88 |
| 1.50 | 15 | 0.91 | 2.26 | 15 | 0.17 | 0.22 | 16 | 21.51 | 21.82 |
| 1.75 | 15 | 2.12 | 5.34 | 14 | 0.40 | 0.64 | 16 | 25.09 | 20.65 |
| 2.00 | 15 | 3.42 | 6.67 | 15 | 0.78 | 1.27 | 16 | 28.42 | 19.39 |
| 2.25 | 15 | 6.68 | 10.50 | 15 | 1.52 | 2.66 | 16 | 32.07 | 18.57 |
| 2.50 | 15 | 8.11 | 10.63 | 15 | 2.77 | 4.15 | 16 | 33.89 | 17.75 |
| 2.75 | 15 | 10.26 | 12.44 | 15 | 4.80 | 5.53 | 16 | 34.38 | 17.30 |
| 3.00 | 16 | 11.87 | 14.56 | 14 | 8.71 | 9.31 | 16 | 36.04 | 17.05 |
| 3.50 | 16 | 19.58 | 15.20 | 16 | 18.86 | 19.99 | 16 | 36.81 | 16.90 |
| 4.00 | 16 | 32.81 | 16.71 | 16 | 28.95 | 21.14 | 16 | 34.65 | 16.31 |
| 6.00 | 16 | 29.35 | 17.37 | 16 | 32.63 | 16.18 | 16 | 25.38 | 13.06 |
| 8.00 | 16 | 21.32 | 11.26 | 16 | 24.23 | 12.58 | 16 | 20.09 | 11.49 |
| 12.00 | 16 | 14.04 | 8.73 | 16 | 16.54 | 9.80 | 16 | 13.12 | 7.91 |
| 24.00 | 16 | 7.36 | 5.37 | 16 | 7.87 | 5.24 | 16 | 6.85 | 4.87 |
| 48.00 | 16 | 2.68 | 2.49 | 16 | 3.08 | 2.48 | 16 | 2.52 | 2.26 |
| 72.00 | 16 | 1.20 | 1.29 | 16 | 1.39 | 1.29 | 16 | 1.15 | 1.24 |
| 96.00 | 13 | 0.85 | 0.81 | 13 | 0.88 | 0.73 | 15 | 0.66 | 0.71 |

TABLE 17
Descriptive statistics of pitolisant PK parameters in serum by treatment.

| | | t_{lag} | tₘₐₓ | Cₘₐₓ | AUC₀₋ₜ | AUC_{0-∞} | kₑ | t_{1/2} | CL/F | Vd/F | Frel |
|---|---|---|---|---|---|---|---|---|---|---|---|
| | | (h) | (h) | (ng/mL) | (h*ng/mL) | (h*ng/mL) | (1/h) | (h) | (L/h) | (L) | (%) |
| A | N | 15 | 15 | 15 | 15 | 15 | 15 | 15 | 15 | 15 | 15 |
| | Mean | 2.42 | 5.07 | 36.04 | 562.69 | 582.09 | 0.04 | 20.10 | 90.41 | 2548.28 | 1.19 |
| | SD | 1.16 | 1.04 | 15.06 | 311.03 | 326.57 | 0.01 | 3.63 | 69.90 | 1939.12 | 0.46 |
| | Min | 0.25 | 4.00 | 8.86 | 122.86 | 125.75 | 0.03 | 11.66 | 30.94 | 777.56 | 0.49 |
| | Median | 2.50 | 6.00 | 35.42 | 490.03 | 494.91 | 0.03 | 20.33 | 71.93 | 2052.24 | 1.08 |
| | Max | 4.00 | 6.03 | 63.79 | 1130.25 | 1150.61 | 0.06 | 25.72 | 283.10 | 7442.03 | 2.21 |
| | CV % | 47.99 | 20.45 | 41.78 | 55.28 | 56.10 | 22.99 | 18.08 | 77.31 | 76.10 | 38.56 |
| | GM | 1.99 | 4.97 | 32.45 | 474.88 | 489.18 | 0.04 | 19.75 | 72.78 | 2073.80 | 1.12 |
| B | N | 15 | 16 | 16 | 15 | 15 | 15 | 15 | 15 | 15 | 15 |
| | Mean | 2.17 | 4.41 | 43.29 | 607.63 | 633.30 | 0.04 | 21.09 | 85.66 | 2382.01 | 1.28 |
| | SD | 0.97 | 1.60 | 23.73 | 358.99 | 375.48 | 0.01 | 8.73 | 66.50 | 1693.15 | 0.54 |
| | Min | 0.00 | 0.00 | 0.00 | 138.84 | 142.56 | 0.01 | 10.89 | 28.45 | 621.21 | 0.81 |
| | Median | 2.50 | 4.00 | 38.24 | 437.96 | 443.12 | 0.03 | 19.87 | 80.34 | 1974.87 | 1.18 |
| | Max | 3.50 | 6.00 | 82.89 | 1230.82 | 1251.34 | 0.06 | 46.67 | 249.72 | 7159.84 | 2.78 |
| | CV % | 44.83 | 36.41 | 54.81 | 59.08 | 59.29 | 32.26 | 41.41 | 77.64 | 71.08 | 42.28 |
| | GM | NC | NC | NC | 503.40 | 522.57 | 0.04 | 19.80 | 68.12 | 1945.54 | 1.19 |
| C | N | 16 | 16 | 16 | 16 | 16 | 16 | 16 | 16 | 16 | 16 |
| | Mean | 1.08 | 4.47 | 37.07 | 531.49 | 555.48 | 0.04 | 19.77 | 113.08 | 2906.79 | 1.03 |
| | SD | 0.84 | 1.09 | 17.06 | 339.19 | 367.28 | 0.01 | 4.18 | 115.15 | 2619.80 | 0.49 |
| | Min | 0.00 | 3.50 | 6.48 | 78.90 | 82.22 | 0.03 | 12.24 | 25.30 | 977.27 | 0.60 |
| | Median | 1.00 | 4.00 | 38.80 | 523.32 | 544.51 | 0.04 | 19.58 | 65.59 | 2016.95 | 0.93 |
| | Max | 2.50 | 6.00 | 65.22 | 1313.17 | 1407.03 | 0.06 | 27.55 | 432.96 | 11067.86 | 2.70 |
| | CV % | 77.47 | 24.33 | 46.02 | 63.82 | 66.12 | 23.07 | 21.12 | 101.83 | 90.13 | 47.69 |
| | GM | NC | 4.35 | 32.09 | 422.45 | 437.63 | 0.04 | 19.35 | 81.35 | 2270.50 | 0.96 |
| D | N | 16 | 16 | 16 | 16 | 16 | 16 | 16 | 16 | 16 | 16 |
| | Mean | 1.31 | 5.00 | 35.97 | 578.64 | 603.22 | 0.04 | 19.70 | 104.80 | 2637.11 | 1.11 |
| | SD | 1.10 | 1.03 | 17.41 | 362.00 | 387.55 | 0.01 | 4.13 | 103.87 | 2324.11 | 0.42 |
| | Min | 0.00 | 4.00 | 9.40 | 84.32 | 87.60 | 0.03 | 13.14 | 25.52 | 1013.69 | 0.41 |
| | Median | 1.13 | 5.00 | 35.59 | 511.23 | 530.74 | 0.04 | 18.79 | 67.19 | 1956.14 | 1.08 |
| | Max | 3.50 | 6.00 | 74.28 | 1297.94 | 1395.12 | 0.05 | 27.72 | 406.40 | 10261.72 | 2.36 |
| | CV % | 83.61 | 20.66 | 48.41 | 62.56 | 64.25 | 21.88 | 20.97 | 99.12 | 88.13 | 38.34 |
| | GM | NC | 4.90 | 31.44 | 457.26 | 473.19 | 0.04 | 19.29 | 75.23 | 2093.25 | 1.04 |
| E | N | 16 | 16 | 16 | 16 | 16 | 16 | 16 | 16 | 16 | 0 |
| | Mean | 0.06 | 2.73 | 42.49 | 560.38 | 581.69 | 0.04 | 20.45 | 110.07 | 3091.62 | NA |
| | SD | 0.11 | 0.74 | 21.19 | 347.12 | 371.76 | 0.01 | 4.10 | 112.98 | 3043.12 | NA |
| | Min | 0.00 | 1.50 | 7.25 | 87.81 | 91.21 | 0.02 | 14.83 | 25.93 | 1088.05 | NA |
| | Median | 0.00 | 2.75 | 38.06 | 470.23 | 483.83 | 0.03 | 20.01 | 73.96 | 1970.98 | NA |
| | Max | 0.25 | 4.00 | 79.36 | 1263.46 | 1372.73 | 0.05 | 29.24 | 390.32 | 11020.35 | NA |
| | CV % | 178.89 | 27.01 | 49.87 | 61.94 | 63.91 | 19.37 | 20.05 | 102.64 | 98.43 | NA |
| | GM | NC | 2.64 | 36.23 | 442.47 | 456.10 | 0.03 | 20.08 | 78.05 | 2260.98 | NA |

NC: Not calculable as at least one value was equal to 0;
NA: Not applicable

TABLE 18
ANOVA Table for bioequivalence assessment.

| Parameter | Source | DF | Type III SS | Mean Square | F Value | Pr > F |
|---|---|---|---|---|---|---|
| Cₘₐₓ (ng/mL) | TREATMENT | 4 | 0.732454 | 0.183113 | 2.66 | 0.0425 |
| | PERIOD | 4 | 0.619678 | 0.154920 | 2.25 | 0.0758 |
| | SUBJID(SEQUENCE) | 6 | 11.149485 | 1.858247 | 26.98 | <.0001 |
| | ERROR: MS(ERROR) | 54 | 3.719883 | 0.068887 | — | — |
| | SEQUENCE | 9 | 9.781660 | 1.086851 | 0.57 | 0.7814 |
| | ERROR | 5.9922 | 11.326888 | 1.890279 | — | — |
| AUC₀₋ₜ | TREATMENT | 4 | 0.467960 | 0.116990 | 2.55 | 0.0498 |
| (h · ng/mL) | PERIOD | 4 | 0.432280 | 0.108070 | 2.35 | 0.0656 |
| | SUBJID(SEQUENCE) | 6 | 19.996800 | 3.332800 | 72.51 | <.0001 |
| | ERROR: MS(ERROR) | 54 | 2.482008 | 0.045963 | — | — |
| | SEQUENCE | 9 | 16.910942 | 1.878994 | 0.55 | 0.7956 |
| | ERROR | 5.9971 | 20.339953 | 3.391637 | — | — |
| AUC_{0-inf} | TREATMENT | 4 | 0.475639 | 0.118910 | 2.59 | 0.0467 |
| (h · ng/mL) | PERIOD | 4 | 0.468442 | 0.117110 | 2.55 | 0.0494 |
| | SUBJID(SEQUENCE) | 6 | 20.707584 | 3.451264 | 75.20 | <.0001 |
| | ERROR: MS(ERROR) | 54 | 2.478261 | 0.045894 | — | — |
| | SEQUENCE | 9 | 17.050881 | 1.894542 | 0.54 | 0.8055 |
| | ERROR | 5.9972 | 21.063483 | 3.512223 | — | — |

Sequence was tested against Subject (nested within sequence) as an error term, other effects were tested against residual error (MS(Error)); MS: Mean-Square; SS: Sum of Square; DF: degrees of freedom.

TABLE 19
Statistical results of bioequivalence assessment.

| | | Reference | | | Ratio (Test/Reference) | | |
|---|---|---|---|---|---|---|---|
| | W-CV | (Treatment E) | Test | | GMR | | Reference |
| Parameter | (%) | GM | Treatment | GM | (Test/Ref.) | GMR 90% CI | Interval |
| Cₘₐₓ (ng/mL) | 26.7 | 36.45 | A | 34.74 | 0.9533 | 0.8121; 1.1189 | 0.8000; 1.2500 |
| | | | B | 42.48 | 1.1654 | 0.9942; 1.3662 | 0.8000; 1.2500 |
| | | | C | 32.58 | 0.8940 | 0.7649; 1.0448 | 0.8000; 1.2500 |
| | | | D | 32.40 | 0.8889 | 0.7593; 1.0406 | 0.8000; 1.2500 |
| AUC₀₋ₜ | 21.7 | 441.80 | A | 505.83 | 1.1449 | 1.0045; 1.3050 | 0.8000; 1.2500 |
| (h · ng/mL) | | | B | 523.50 | 1.1849 | 1.0407; 1.3492 | 0.8000; 1.2500 |
| | | | C | 425.74 | 0.9636 | 0.8484; 1.0946 | 0.8000; 1.2500 |
| | | | D | 466.42 | 1.0557 | 0.9282; 1.2008 | 0.8000; 1.2500 |
| AUC_{0-inf} | 21.7 | 454.84 | A | 520.61 | 1.1446 | 1.0043; 1.3045 | 0.8000; 1.2500 |
| (h · ng/mL) | | | B | 543.26 | 1.1944 | 1.0491; 1.3598 | 0.8000; 1.2500 |
| | | | C | 440.64 | 0.9688 | 0.8530; 1.1003 | 0.8000; 1.2500 |
| | | | D | 482.42 | 1.0606 | 0.9326; 1.2062 | 0.8000; 1.2500 |

GM: geometric mean;
GMR: geometric mean ratio;
CI: confidence interval;
BE: bioequivalence;
W-CV: within subject CV (%)

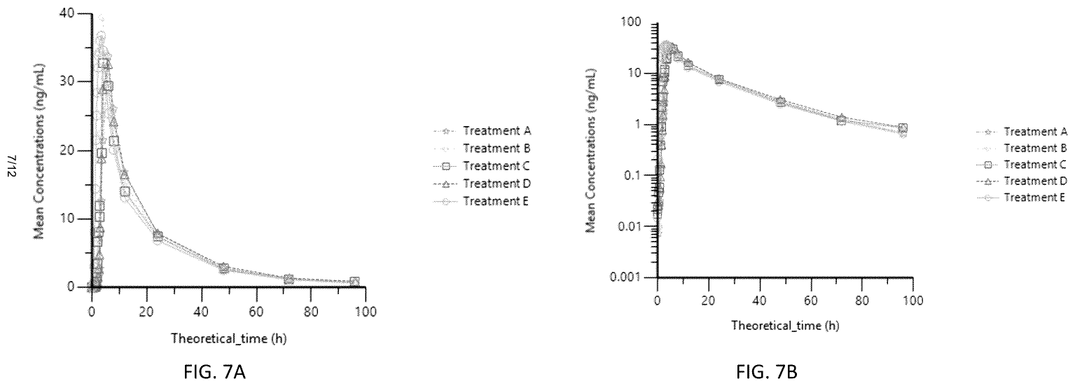

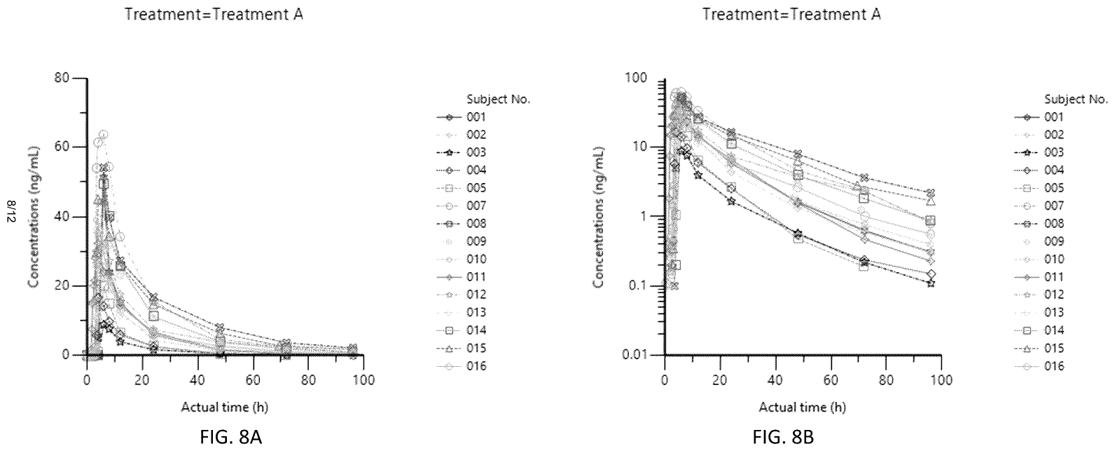

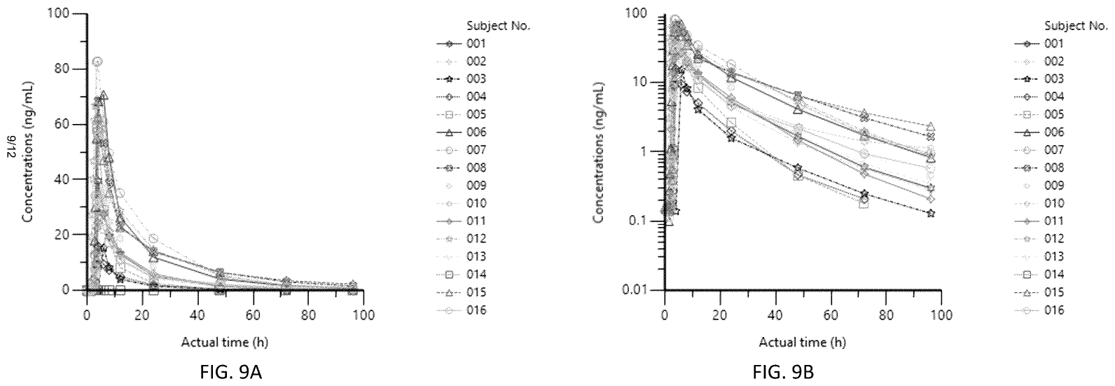

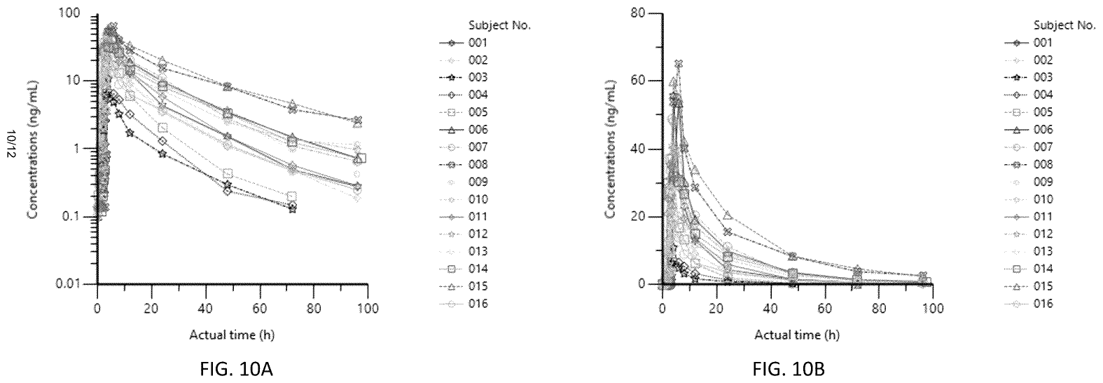

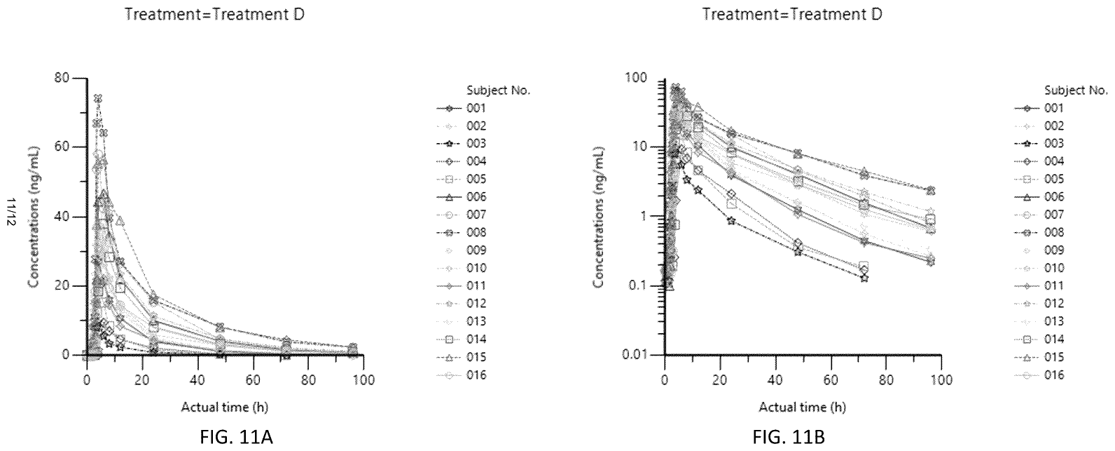

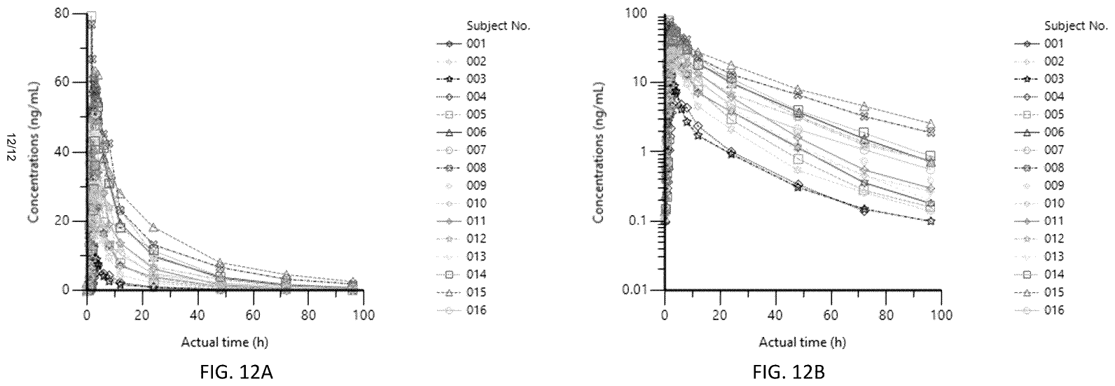

Graphs depicting pharmacokinetic profiles are provided as FIGS. 7A, 7B, 8A, 8B, 9A, 9B, 10A, 10B, 11A, 11B, 12A, and 12B.

Based on the pharmacokinetic data obtained in this clinical study it was discovered that the exemplary dose forms of the present application (Treatments A-D) showed a meaningful improvement over WAKIX®, with at least about 20% increase in relative bioavailability and a decrease in the variability, as compared to the reference Treatment E (WAKIX® at a dose strength of 35.6 mg, which is the highest labeled dose for WAKIX®).

### EQUIVALENTS

Those skilled in the art will recognize or be able to ascertain using no more than routine experimentation many equivalents to the specific embodiments disclosed herein. Those of ordinary skill in the art will appreciate that various changes or modifications to this description may be made without departing from the spirit or scope of the present disclosure, as defined in the following claims.

What is claimed is:

1. An oral dosage form comprising:
a core; and
a delayed-release coating that surrounds the core,
wherein the core comprises pitolisant or a pharmaceutically acceptable salt, solvate, or hydrate thereof, an alkaline agent, and optionally one or more pharmaceutically acceptable excipients.

2. The dosage form of claim 1, wherein the core comprises pitolisant monohydrochloride.

3. The dosage form of claim 1 or 2, wherein the alkaline agent is selected from the group consisting of magnesium carbonate, potassium bicarbonate, and sodium carbonate.

4. The dosage form of any one of the preceding claims, wherein the delayed-release coating comprises a polymer, optionally wherein the polymer comprises an ionizable functional group (e.g., a carboxylic acid group).

5. The dosage form of any one of the preceding claims, wherein the delayed-release coating comprises a cellulosic material (e.g., an alkylcellulose), an acrylic or acrylate polymer, or a methacrylic or methacrylate copolymer (e.g., an anionic methacrylate copolymer).

6. The dosage form of any one of the preceding claims, wherein the delayed-release coating comprises ACRYL-EZE®.

7. The dosage form of any one of the preceding claims, wherein the delayed-release coating comprises a plasticizer.

8. The dosage form of claim 7, wherein the plasticizer comprises polyethylene glycol (PEG), e.g., PEG8000, or triethyl citrate.

9. The dosage form of any one of the preceding claims, further comprising an anti-moisture barrier.

10. The dosage form of claim 9, wherein the anti-moisture barrier is positioned between the core and the delayed-release coating.

11. The dosage form of claim 9 or 10, wherein the anti-moisture barrier comprises an OPADRY® polymer, e.g., OPADRY® amb II.

12. The dosage form of any one of the preceding claims, comprising between about 1 mg and about 100 mg pitolisant or a pharmaceutically acceptable salt, solvate, or hydrate thereof (e.g., between about 1 mg and about 80 mg, between about 1 mg and about 40 mg, between about 1 mg and about 40 mg, between about 20 mg and about 40 mg, between about 40 mg and about 60 mg, or between about 50 mg and about 70 mg).

13. The dosage form of any one of the preceding claims, comprising about 40 mg of pitolisant or a pharmaceutically acceptable salt, solvate, or hydrate thereof.

14. The dosage form of any one of the preceding claims, comprising about 7.5 mg or about 30 mg of an alkaline agent.

15. The dosage form of any one of the preceding claims, comprising at least one pharmaceutically acceptable excipient.

16. The dosage form of claim 15, wherein the pharmaceutically acceptable excipient is selected from the group consisting of lactose, microcrystalline cellulose, croscarmellose sodium, magnesium stearate, and colloidal silica (e.g., anhydrous colloidal silica).

17. The dosage form of any one of the preceding claims, wherein the pitolisant or a pharmaceutically acceptable salt, solvate, or hydrate thereof is crystalline pitolisant monohydrochloride.

18. The dosage form of any one of the preceding claims, wherein the core comprises (e.g., consists essentially of, e.g., consists of) pitolisant monohydrochloride, an alkaline agent, lactose, microcrystalline cellulose, croscarmellose sodium, magnesium stearate, and colloidal silica (e.g., anhydrous colloidal silica).

19. The dosage form of claim 18, wherein the core is surrounded by an anti-moisture barrier that comprises (e.g., consists essentially of, e.g., consists of) a PVA-based polymer, e.g., OPADRY® amb II.

20. The dosage form of claim 19, wherein the core surrounded by the anti-moisture barrier is further surrounded by a delayed-release coating that comprises (e.g., consists essentially of, e.g., consists of) a copolymer of methacrylic acid and ethyl acrylate, e.g., ACRYL-EZE®.

21. The dosage form of any one of the preceding claims, wherein the pitolisant or a pharmaceutically acceptable salt, solvate, or hydrate thereof has an X-ray diffractogram that comprises characteristic peaks (2θ) at 11.2°, 19.9°, 20.7°, and 34.10 (0.2°).

22. The dosage form of any one of claims 1-20, wherein the pitolisant or a pharmaceutically acceptable salt, solvate, or hydrate thereof has an X-ray diffractogram that comprises characteristic peaks (2θ) at 11.2°, 15.4°, 16.3°, 16.9°, 17.8°, 19.9°, 20.7°, 21.0°, 21.8°, 22.6°, 24.5°, 24.6°, 25.0°, 25.5°, 26.3°, 28.3°, 30.3°, 34.1°, 35.8°, 40.0°, and 46.0° (±0.2°).

23. The dosage form of any one of the preceding claims, wherein the oral dosage form is a tablet.

24. The dosage form of any one of the preceding claims, wherein the dosage form is bioequivalent to a dosage form comprising pitolisant or a pharmaceutically acceptable salt, solvate, or hydrate thereof, in the same amount, that does not comprise a delayed release coating (e.g., WAKIX®), or wherein the dosage form provides a superior bioavailability of pitolisant (e.g., an increase in bioavailability of about 5%, about 10%, about 20%, or greater).

25. The dosage form of any one of the preceding claims, wherein the dosage form provides a superior bioavailability of pitolisant (e.g., an increase in bioavailability of about 5%, about 10%, about 20%, or greater) compared to a dosage form comprising pitolisant or a pharmaceutically acceptable salt, solvate, or hydrate thereof (e.g., in the same amount) that does not comprise a delayed-release coating (e.g., WAKIX®) or an alkaline agent.

26. A method of treating a disease or disorder, comprising orally administering to a subject in need thereof an oral dosage form of any one claims 1-25.

27. The method of claim 26, wherein the disease or disorder is fatigue.

28. The method of claim 26, wherein the disease or disorder is a sleep disorder.

29. The method of claim 26 or 28, wherein the disease or disorder is excessive daytime sleepiness (EDS), cataplexy, narcolepsy, sleep apnea (e.g., obstructive sleep apnea), sleep induced apnea, or diurnal somnolence.

30. The method of any one of claims 26, 28, or 29, wherein the disease or disorder is excessive daytime sleepiness (EDS).

31. The method of any one of claims 26, 28, or 29, wherein the disease or disorder is cataplexy.

32. The method of any one of claims 26-30, wherein the subject has narcolepsy (e.g., the subject is an adult with narcolepsy).

33. A dosage form (e.g., oral dosage form) of any one of claims 1-25, for use in the treatment of a disease or disorder, optionally, wherein the disease or disorder is fatigue, excessive daytime sleepiness (EDS), cataplexy, narcolepsy, sleep apnea (e.g., obstructive sleep apnea), sleep induced apnea, or diurnal somnolence.

34. The dosage form for use of claim 33, wherein the disease or disorder is in a subject with narcolepsy (e.g., an adult subject with narcolepsy).

35. Use of an oral dosage form of any one of claims 1-25 for the manufacture of a medicament for the treatment of a disease or disorder, optionally, wherein the disease or disorder is fatigue, excessive daytime sleepiness (EDS), cataplexy, narcolepsy, sleep apnea (e.g., obstructive sleep apnea), sleep induced apnea, or diurnal somnolence.

36. The use of claim 35, wherein the disease or disorder is in a subject with narcolepsy (e.g., an adult subject with narcolepsy).

37. A method of making an oral dosage form of any one of claims 1-25.

38. The method of claim 37, comprising the steps of:
a) blending pitolisant or a pharmaceutically acceptable salt, solvate, or hydrate thereof, and an alkaline agent (e.g., magnesium carbonate, potassium bicarbonate, or sodium carbonate), with one or more pharmaceutically acceptable excipients (e.g., lactose, microcrystalline cellulose, croscarmellose sodium, magnesium stearate, colloidal silica (e.g., anhydrous colloidal silica) or a combination thereof), to provide a blend;
b) tableting the blend (e.g., using a tableting press) to provide a core tablet;
c) optionally, coating the core tablet with an anti-moisture barrier, to provide an anti-moisture coated tablet; and
d) coating the core tablet of step (b) or the anti-moisture coated tablet of step (c) with a delayed-release coating, to provide a delayed-release coated tablet.

39. The method of claim 38, wherein the anti-moisture barrier comprises polyvinyl alcohol (PVA) or HPMC, e.g., an OPADRY® polymer, e.g., OPADRY® amb II.

40. The method of claim 38 or 39, wherein the delayed-release coating comprises a copolymer of methacrylic acid and ethyl acrylate, e.g., ACRYL-EZE®.

41. The method of any one of claims 38-40, wherein the delayed-release coating further comprises a plasticizer, e.g., triethyl citrate or PEG (e.g., PEG8000).

42. A pharmaceutical composition comprising an oral dosage form of any one claims 1-25 for oral administration to a subject in need thereof.

43. An oral dosage form of any one claims 1-25 for use in treating fatigue.

44. An oral dosage form of any one claims 1-25 for use in treating a disease or disorder selected from sleep disorders.

45. The oral dosage form for use according to claim 44 wherein the sleep disorder is excessive daytime sleepiness (EDS), cataplexy, narcolepsy, sleep apnea (e.g., obstructive sleep apnea), sleep induced apnea, or diurnal somnolence.

46. The oral dosage form for use according to claim 44 or 45, wherein the disease or disorder is excessive daytime sleepiness (EDS).

47. The oral dosage form for use according to claim 44 or 45, wherein the disease or disorder is cataplexy.

48. The oral dosage form for use according to any one of claims 44 to 47, wherein the subject has narcolepsy (e.g., the subject is an adult with narcolepsy).
